(19) United States
(12) Patent Application Publication
(10) Pub. No.: US 2025/0352064 A1
(43) Pub. Date: Nov. 20, 2025

(54) SYSTEMS AND METHODS FOR OCT-GUIDED TREATMENT OF A PATIENT

(71) Applicant: Spryte Medical, Inc., Bedford, MA (US)

(72) Inventors: Christopher C. Petroff, Groton, MA (US); Christopher L. Petersen, Carlisle, MA (US); David W. Kolstad, Carlisle, MA (US); Giovanni J. Ughi, Arlington, MA (US); Lindsy M. Peterson, Woburn, MA (US); Benjamin Duncan, Watertown, MA (US); R. Maxwell Flaherty, Topsfield, MA (US); J. Christopher Flaherty, Nottingham, NH (US)

(21) Appl. No.: 19/032,699

(22) Filed: Jan. 21, 2025

Related U.S. Application Data
(63) Continuation of application No. 17/603,689, filed on Oct. 14, 2021, now Pat. No. 12,239,412.
(60) Provisional application No. 62/850,945, filed on May 21, 2019.
(60) Provisional application No. 62/906,353, filed on Sep. 26, 2019.
(60) Provisional application No. 63/017,258, filed on Apr. 29, 2020.

(51) Int. Cl. A61B 5/00; A61B 90/00

(52) U.S. Cl. CPC A61B 5/0066; A61B 5/0044; A61B 5/0084; A61B 90/37; A61B 2090/3735; A61B 2090/376

(57) ABSTRACT

Provided herein are imaging systems for a patient comprising an imaging probe and an imaging assembly. The imaging probe comprises: an elongate shaft comprising a proximal end, a distal portion, and a lumen extending between the proximal end and the distal portion; a rotatable optical core comprising a proximal end and a distal end, wherein at least a portion of the rotatable optical core is positioned within the lumen of the elongate shaft; and an optical assembly positioned proximate the distal end of the rotatable optical core, the optical assembly configured to direct light to tissue and collect reflected light from the tissue. The imaging assembly is constructed and arranged to optically couple to the imaging probe. The imaging assembly is configured to emit light into the imaging probe and receive the reflected light collected by the optical assembly. The system is configured to provide treatment information, wherein the treatment information is used by an operator to plan a treatment and/or predict a treatment outcome.

### Related Applications

This application is a continuation application of U.S. patent application Ser. No. 17/603,689, titled “Systems and Methods for OCT-Guided Treatment of a Patient”, filed Oct. 14, 2021, Publication Number 2022-0061670, published Mar. 3, 2022, which is a U.S. National Stage entry of International PCT patent Application Serial Number PCT/US2020/033953, titled “Systems and Methods for OCT-Guided Treatment of a Patient”, filed May 21, 2020, Publication Number WO 2020/237024, published Nov. 26, 2020, which claims priority to U.S. Provisional Application Ser. No. 62/850,945, titled “OCT-Guided Treatment of a Patient”, filed May 21, 2019, and U.S. Provisional Application Ser. No. 62/906,353, titled “OCT-Guided Treatment of a Patient”, filed Sep. 26, 2019, and U.S. Provisional Application Ser. No. 63/017,258, titled “Imaging System”, filed Apr. 29, 2020, the content of each of which is incorporated by reference in its entirety.

This application is related to U.S. Provisional Application Ser. No. 62/148,355, titled “Micro-Optic Probes for Neurology”, filed Apr. 16, 2015, the content of which is incorporated by reference in its entirety.

This application is related to United States Provisional Application Ser. No. 62/322,182, titled “Micro-Optic Probes for Neurology”, filed Apr. 13, 2016, the content of which is incorporated by reference in its entirety.

This application is related to International PCT Patent Application Serial Number PCT/US2016/027764, titled “Micro-Optic Probes for Neurology” filed Apr. 15, 2016, Publication Number WO 2016/168605, published Oct. 20, 2016, the content of which is incorporated by reference in its entirety.

This application is related to U.S. patent application Ser. No. 15/566,041, titled “Micro-Optic Probes for Neurology”, filed Oct. 12, 2017, United States Publication Number 2018-0125372, published May 10, 2018, the content of which is incorporated by reference in its entirety.

This application is related to United States Provisional Application Ser. No. 62/212,173, titled “Imaging System Includes Imaging Probe and Delivery Devices”, filed Aug. 31, 2015, the content of which is incorporated by reference in its entirety.

This application is related to U.S. Provisional Application Ser. No. 62/368,387, titled “Imaging System Includes Imaging Probe and Delivery Devices”, filed Jul. 29, 2016, the content of which is incorporated by reference in its entirety.

This application is related to International PCT Patent Application Serial Number PCT/US2016/049415, titled “Imaging System Includes Imaging Probe and Delivery Devices”, filed Aug. 30, 2016, Publication Number WO 2017/040484, published Mar. 9, 2017, the content of which is incorporated by reference in its entirety.

This application is related to U.S. patent application Ser. No. 15/751,570, titled “Imaging System Includes Imaging Probe and Delivery Devices”, filed Feb. 9, 2018, U.S. Pat. No. 10,631,718, issued Apr. 28, 2020, the content of which is incorporated by reference in its entirety.

This application is related to U.S. patent application Ser. No. 16/820,991, titled “Imaging System Includes Imaging Probe and Delivery Devices”, filed Mar. 17, 2020, Publication Number 2021-0045622, published Feb. 18, 2021, the content of which is incorporated by reference in its entirety.

This application is related to U.S. Provisional Application Ser. No. 62/591,403, titled “Imaging System”, filed Nov. 28, 2017, the content of which is incorporated by reference in its entirety.

This application is related to United States Provisional Application Ser. No. 62/671,142, titled “Imaging System”, filed May 14, 2018, the content of which is incorporated by reference in its entirety.

This application is related to International PCT Patent Application Serial Number PCT/US2018/062766, titled “Imaging System”, filed Nov. 28, 2018, Publication Number WO 2019/108598, published Jun. 6, 2019, the content of which is incorporated by reference in its entirety.

This application is related to United States Provisional Application Ser. No. 62/732,114, titled “Imaging System with Optical Pathway”, filed Sep. 17, 2018, the content of which is incorporated by reference in its entirety.

This application is related to International PCT Patent Application Serial Number PCT/US2019/051447, titled “Imaging System with Optical Pathway”, filed Sep. 17, 2019, Publication Number WO 2020/061001, published Mar. 26, 2020, the content of which is incorporated by reference in its entirety.

This application is related to U.S. Provisional Application Ser. No. 62/840,450, titled “Imaging Probe with Fluid Pressurization Element”, filed Apr. 30, 2019, the content of which is incorporated by reference in its entirety.

This application is related to International PCT Patent Application Serial Number PCT/US2020/030616, titled “Imaging Probe with Fluid Pressurization Element”, filed Apr. 30, 2020, Publication Number WO 2020/223433, published Nov. 5, 2020, the content of which is incorporated by reference in its entirety.

### FIELD OF THE INVENTION

The present invention relates generally to optical coherence tomography (OCT) imaging systems, and in particular, to systems that provide guidance for therapeutic treatment of a patient.

### BACKGROUND

Imaging probes have been commercialized for imaging various internal locations of a patient, such as an intravascular probe for imaging a patient's heart. Current imaging probes are limited in their ability to reach certain anatomical locations due to their size and rigidity. Current imaging probes are inserted over a guidewire, which can compromise their placement and limit use of one or more delivery catheters through which the imaging probe is inserted. There is a need for imaging systems that include probes with reduced diameter and high flexibility, as well as systems with one or more delivery devices compatible with these improved imaging probes.

### SUMMARY

According to an aspect of the present inventive concepts, an imaging system for a patient comprises an imaging probe, an optical assembly, and an imaging assembly. The imaging probe comprises: an elongate shaft comprising a proximal end, a distal portion, and a lumen extending between the proximal end and the distal portion; a rotatable optical core comprising a proximal end and a distal end, and at least a portion of the rotatable optical core is positioned within the lumen of the elongate shaft. The optical assembly is positioned proximate the distal end of the rotatable optical core and is configured to direct light to tissue and collect reflected light from the tissue. The imaging assembly is constructed and arranged to optically couple to the imaging probe and is configured to emit light into the imaging probe and receive the reflected light collected by the optical assembly. The system can be configured to provide treatment information, and the treatment information can be used by an operator to plan a treatment and/or predict a treatment outcome.

In some embodiments, the imaging probe further comprises a damping fluid positioned between the elongate shaft and the rotatable optical core and configured to reduce non-uniform rotation of the optical assembly. The imaging probe can further comprise a fluid pressurization element configured to increase the pressure of the damping fluid to reduce the presence of bubbles proximate the optical assembly.

In some embodiments, the treatment information is based on OCT data gathered by the imaging probe. The system can further comprise a second imaging device configured to gather non-OCT data, and the treatment information can be further based on the non-OCT data. The second imaging device can be configured to gather non-OCT data comprising angiography data.

In some embodiments, the system is configured to produce an assessment of disease severity. The assessment can comprise a quantified assessment and/or a qualitative assessment.

In some embodiments, the system is configured to accurately capture a clot of 5 μm or more, 10 μm or more, or 30 μm or more.

The technology described herein, along with the attributes and attendant advantages thereof, will best be appreciated and understood in view of the following detailed description taken in conjunction with the accompanying drawings in which representative embodiments are described by way of example.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

FIG. 1 illustrates a schematic view of an imaging system comprising an imaging probe and independent retraction and rotation assemblies, consistent with the present inventive concepts.

FIG. 1A illustrates a schematic view of an imaging system comprising an imaging probe operably attachable to a patient interface module, and an independent pullback module operably attachable to the patient interface module and the imaging probe, consistent with the present inventive concepts.

FIG. 1B illustrates a schematic view of an imaging system comprising an imaging probe operably attachable to a module comprising a first connector for attaching to a rotation motive element and a second connector for attaching to a retraction motive element, consistent with the present inventive concepts.

FIG. 2A illustrates a perspective view of connectors being attached to a patient interface module, consistent with the present inventive concepts.

FIG. 2B illustrates a perspective view of a pullback housing, consistent with the present inventive concepts.

FIG. 3 illustrates a perspective view of connectors being attached to a patient interface module, consistent with the present inventive concepts.

FIG. 4 illustrates a flow chart of a method of planning a treatment procedure for a patient, consistent with the present inventive concepts.

FIG. 5 illustrates a flow chart of a method of planning a treatment procedure, performing a treatment, and assessing the treatment, consistent with the present inventive concepts.

FIG. 6 illustrates a flowchart of a method of planning a treatment procedure for a patient, consistent with the present inventive concepts.

FIG. 7 illustrates a full Navier-Stokes simulation of flow through an artery, and the FFR calculated through the length of the artery, consistent with the present inventive concepts.

FIG. 8 illustrates a graph of a velocity profile through a pipe.

FIG. 9 illustrates the velocity and shear stress of blood near a vessel wall.

FIG. 10 illustrates a vessel flow schematic, consistent with the present inventive concepts.

FIG. 11 illustrates a sequence of steps for performing a pullback procedure, consistent with the present inventive concepts.

FIGS. 12 and 12A illustrate a schematic view of the distal portion of an imaging probe and delivery catheter, and a magnified view of the components within circle M2, respectively, consistent with the present inventive concepts.

FIGS. 12B and 12C illustrate a schematic view of the distal portion of an imaging probe showing a fluid flow pattern, and a fluid flow simulation, respectively, consistent with the present inventive concepts.

FIG. 13 illustrates the principles of system imaging in an intracranial vessel.

FIG. 14A illustrates a fluoroscopic image of a tortuous path through a flexed forelimb model of brachial porcine artery.

FIG. 14B illustrates system imaging of the flexed forelimb model of brachial porcine artery.

FIG. 14C illustrates the eccentric position of the imaging probe in the flexed forelimb model of brachial porcine artery.

FIG. 15A illustrates vessel fluoroscopy of a stented swine internal maxillary artery.

FIG. 15B illustrates a system three-dimensional cutaway rendering of the stented swine internal maxillary artery.

FIG. 15C illustrates a cross-sectional system imaging of the stented swine internal maxillary artery.

FIG. 15D illustrates a cross-sectional system imaging of a flow diverter proximal edge of the stented swine internal maxillary artery.

FIG. 16A illustrates a side-branch thrombosis visible on a system image.

FIG. 16B illustrates a flow-diverter malapposition with a maximum severity of approximately 400 μm visible on the system image.

FIG. 16C illustrates thrombosis on the ostium of a large side-branch.

FIG. 17A illustrates an endoscopic view of system volumetric microscopy data.

FIG. 17B illustrates a first side-branch free of clots and an FDS well apposed to the parent artery.

FIG. 17C illustrates a second branch with FDS struts that are embedded by a clot.

FIG. 18A illustrates system volumetric microscopy data showing an ICS partially overlapping with the distal end of an FDS.

FIG. 18B illustrates system microscopy showing individual vessel layers comprising a bright tunica intima, a low-scattering tunica media, and the tunica adventitia.

FIG. 18C illustrates a thrombus dislodged from the surface of an imaging probe floating inside a large branch.

FIG. 18D illustrates thrombus formations ranging between 100 μm and 200 μm in thickness and distributed over the FDS surface.

FIG. 18E illustrates a semi-occlusive clot formation in correspondence of a significant malapposition (e.g. at least 500 μm) and visible over the proximal end of the FDS.

FIG. 19A illustrates system imaging of a fibrotic plaque and corresponding trichrome.

FIG. 19B illustrates a moyat's staining of the fibrotic plaque and corresponding trichrome.

FIG. 19C illustrates fibrotic tissue characterized by the imaging system as a region of homogenous signal, resulting from elevated backscattering and low optical attenuation coefficients.

FIG. 19D illustrates system imaging of a necrotic core plaque and corresponding

H&E.

FIG. 19E illustrates moyat's staining of the necrotic core plaque and

corresponding H&E.

FIG. 19F illustrates a necrotic core plaque characterized on system images as an area with poorly delineated borders followed by an elevated optical attenuation coefficient within an atherosclerotic plaque.

FIGS. 20A and B illustrate intracranial fibrocalcific plaques in a segment of an intradural vertebral artery.

FIG. 21 illustrates a bench model of a full, patient-specific circle of Willis.

FIG. 22A illustrates blood obscuring a field of view.

FIG. 22B illustrates partial clearance of blood from the field of view.

FIG. 22C illustrates complete clearance of blood from the field of view.

FIG. 23A illustrates system imaging of a stented segment in a swine common carotid artery with a maximum diameter of 5.9 mm following deployment of a Precise Pro Rx Carotid Stent System (Cordis).

FIG. 23B illustrates a three-dimensional system rendering of the swine common carotid artery.

FIG. 23C illustrates system cross sectional imaging of the swine common carotid artery.

FIG. 23D illustrates an external elastic membrane (EEL) of the Precise Pro Rx Carotid Stent System.

### DETAILED DESCRIPTION OF THE DRAWINGS

Reference will now be made in detail to the present embodiments of the technology, examples of which are illustrated in the accompanying drawings. Similar reference numbers may be used to refer to similar components. However, the description is not intended to limit the present disclosure to particular embodiments, and it should be construed as including various modifications, equivalents, and/or alternatives of the embodiments described herein.

It will be understood that the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) when used herein, specify the presence of stated features, integers, steps, operations, elements, and/or components, but do not preclude the presence or addition of one or more other features, integers, steps, operations, elements, components, and/or groups thereof.

It will be further understood that, although the terms first, second, third, etc. may be used herein to describe various limitations, elements, components, regions, layers and/or sections, these limitations, elements, components, regions, layers and/or sections should not be limited by these terms. These terms are only used to distinguish one limitation, element, component, region, layer or section from another limitation, element, component, region, layer or section. Thus, a first limitation, element, component, region, layer or section discussed below could be termed a second limitation, element, component, region, layer or section without departing from the teachings of the present application.

It will be further understood that when an element is referred to as being “on”, “attached”, “connected” or “coupled” to another element, it can be directly on or above, or connected or coupled to, the other element, or one or more intervening elements can be present. In contrast, when an element is referred to as being “directly on”, “directly attached”, “directly connected” or “directly coupled” to another element, there are no intervening elements present. Other words used to describe the relationship between elements should be interpreted in a like fashion (e.g. “between” versus “directly between,” “adjacent” versus “directly adjacent,” etc.).

It will be further understood that when a first element is referred to as being “in”, “on” and/or “within” a second element, the first element can be positioned: within an internal space of the second element, within a portion of the second element (e.g. within a wall of the second element); positioned on an external and/or internal surface of the second element; and combinations of one or more of these.

As used herein, the term “proximate”, when used to describe proximity of a first component or location to a second component or location, is to be taken to include one or more locations near to the second component or location, as well as locations in, on and/or within the second component or location. For example, a component positioned proximate an anatomical site (e.g. a target tissue location), shall include components positioned near to the anatomical site, as well as components positioned in, on and/or within the anatomical site.

Spatially relative terms, such as “beneath,” “below,” “lower,” “above,” “upper” and the like may be used to describe an element and/or feature's relationship to another element(s) and/or feature(s) as, for example, illustrated in the figures. It will be further understood that the spatially relative terms are intended to encompass different orientations of the device in use and/or operation in addition to the orientation depicted in the figures. For example, if the device in a figure is turned over, elements described as “below” and/or “beneath” other elements or features would then be oriented “above” the other elements or features. The device can be otherwise oriented (e.g. rotated 90 degrees or at other orientations) and the spatially relative descriptors used herein interpreted accordingly.

The terms “reduce”, “reducing”, “reduction” and the like, where used herein, are to include a reduction in a quantity, including a reduction to zero. Reducing the likelihood of an occurrence shall include prevention of the occurrence. Correspondingly, the terms “prevent”, “preventing”, and “prevention” shall include the acts of “reduce”, “reducing”, and “reduction”, respectively.

The term “and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

The term “one or more”, where used herein can mean one, two, three, four, five, six, seven, eight, nine, ten, or more, up to any number.

The terms “and combinations thereof” and “and combinations of these” can each be used herein after a list of items that are to be included singly or collectively. For example, a component, process, and/or other item selected from the group consisting of: A; B; C; and combinations thereof, shall include a set of one or more components that comprise: one, two, three or more of item A; one, two, three or more of item B; and/or one, two, three, or more of item C.

In this specification, unless explicitly stated otherwise, “and” can mean “or”, and “or” can mean “and”. For example, if a feature is described as having A, B, or C, the feature can have A, B, and C, or any combination of A, B, and C. Similarly, if a feature is described as having A, B, and C, the feature can have only one or two of A, B, or C.

As used herein, when a quantifiable parameter is described as having a value “between” a first value X and a second value Y, it shall include the parameter having a value of: at least X, no more than Y, and/or at least X and no more than Y. For example, a length of between 1 and 10 shall include a length of at least 1 (including values greater than 10), a length of less than 10 (including values less than 1), and/or values greater than 1 and less than 10.

The expression “configured (or set) to” used in the present disclosure may be used interchangeably with, for example, the expressions “suitable for”, “having the capacity to”, “designed to”, “adapted to”, “made to” and “capable of” according to a situation. The expression “configured (or set) to” does not mean only “specifically designed to” in hardware. Alternatively, in some situations, the expression “a device configured to” may mean that the device “can” operate together with another device or component.

As used herein, the terms “about” or “approximately” shall refer to +30%.

As used herein, the term “threshold” refers to a maximum level, a minimum level, and/or range of values correlating to a desired or undesired state. In some embodiments, a system parameter is maintained above a minimum threshold, below a maximum threshold, within a threshold range of values, and/or outside a threshold range of values, such as to cause a desired effect (e.g. efficacious therapy) and/or to prevent or otherwise reduce (hereinafter “prevent”) an undesired event (e.g. a device and/or clinical adverse event). In some embodiments, a system parameter is maintained above a first threshold (e.g. above a first temperature threshold to cause a desired therapeutic effect to tissue) and below a second threshold (e.g. below a second temperature threshold to prevent undesired tissue damage). In some embodiments, a threshold value is determined to include a safety margin, such as to account for patient variability, system variability, tolerances, and the like. As used herein, “exceeding a threshold” relates to a parameter going above a maximum threshold, below a minimum threshold, within a range of threshold values and/or outside of a range of threshold values.

As described herein, “room pressure” shall mean pressure of the environment surrounding the systems and devices of the present inventive concepts. Positive pressure includes pressure above room pressure or simply a pressure that is greater than another pressure, such as a positive differential pressure across a fluid pathway component such as a valve. Negative pressure includes pressure below room pressure or a pressure that is less than another pressure, such as a negative differential pressure across a fluid component pathway such as a valve. Negative pressure can include a vacuum but does not imply a pressure below room pressure. As used herein, the term “vacuum” can be used to refer to a full or partial vacuum, or any negative pressure as described herein.

The term “diameter” where used herein to describe a non-circular geometry is to be taken as the diameter of a hypothetical circle approximating the geometry being described. For example, when describing a cross section, such as the cross section of a component, the term “diameter” shall be taken to represent the diameter of a hypothetical circle with the same cross sectional area as the cross section of the component being described.

The terms “major axis” and “minor axis” of a component where used herein are the length and diameter, respectively, of the smallest volume hypothetical cylinder which can completely surround the component.

As used herein, the term “functional element” is to be taken to include one or more elements constructed and arranged to perform a function. A functional element can comprise a sensor and/or a transducer. In some embodiments, a functional element is configured to deliver energy and/or otherwise treat tissue (e.g. a functional element configured as a treatment element). Alternatively or additionally, a functional element (e.g. a functional element comprising a sensor) can be configured to record one or more parameters, such as a patient physiologic parameter; a patient anatomical parameter (e.g. a tissue geometry parameter); a patient environment parameter; and/or a system parameter. In some embodiments, a sensor or other functional element is configured to perform a diagnostic function (e.g. to gather data used to perform a diagnosis). In some embodiments, a functional element is configured to perform a therapeutic function (e.g. to deliver therapeutic energy and/or a therapeutic agent). In some embodiments, a functional element comprises one or more elements constructed and arranged to perform a function selected from the group consisting of: deliver energy; extract energy (e.g. to cool a component); deliver a drug or other agent; manipulate a system component or patient tissue; record or otherwise sense a parameter such as a patient physiologic parameter or a system parameter; and combinations of one or more of these. A functional element can comprise a fluid and/or a fluid delivery system. A functional element can comprise a reservoir, such as an expandable balloon or other fluid-maintaining reservoir. A “functional assembly” can comprise an assembly constructed and arranged to perform a function, such as a diagnostic and/or therapeutic function. A functional assembly can comprise an expandable assembly. A functional assembly can comprise one or more functional elements.

The term “transducer” where used herein is to be taken to include any component or combination of components that receives energy or any input, and produces an output. For example, a transducer can include an electrode that receives electrical energy, and distributes the electrical energy to tissue (e.g. based on the size of the electrode). In some configurations, a transducer converts an electrical signal into any output, such as: light (e.g. a transducer comprising a light emitting diode or light bulb), sound (e.g. a transducer comprising a piezo crystal configured to deliver ultrasound energy); pressure (e.g. an applied pressure or force); heat energy; cryogenic energy; chemical energy; mechanical energy (e.g. a transducer comprising a motor or a solenoid); magnetic energy; and/or a different electrical signal (e.g. different than the input signal to the transducer). Alternatively or additionally, a transducer can convert a physical quantity (e.g. variations in a physical quantity) into an electrical signal. A transducer can include any component that delivers energy and/or an agent to tissue, such as a transducer configured to deliver one or more of: electrical energy to tissue (e.g. a transducer comprising one or more electrodes); light energy to tissue (e.g. a transducer comprising a laser, light emitting diode and/or optical component such as a lens or prism); mechanical energy to tissue (e.g. a transducer comprising a tissue manipulating element); sound energy to tissue (e.g. a transducer comprising a piezo crystal); chemical energy; electromagnetic energy; magnetic energy; and combinations of one or more of these.

As used herein, the term “fluid” can refer to a liquid, gas, gel, or any flowable material, such as a material which can be propelled through a lumen and/or opening.

As used herein, the term “material” can refer to a single material, or a combination of two, three, four, or more materials.

As used herein, the term “lesion” comprises a segment of a blood vessel (e.g. an artery) that is in an undesired state. As used herein, lesion shall include a narrowing of a blood vessel (e.g. a stenosis), and/or a segment of a blood vessel, with or without narrowing, that includes a buildup of calcium, lipids, cholesterol, and/or other plaque.

It is appreciated that certain features of the invention, which are, for clarity, described in the context of separate embodiments, may also be provided in combination in a single embodiment. Conversely, various features of the invention which are, for brevity, described in the context of a single embodiment, may also be provided separately or in any suitable sub-combination. For example, it will be appreciated that all features set out in any of the claims (whether independent or dependent) can be combined in any given way.

It is to be understood that at least some of the figures and descriptions of the invention have been simplified to focus on elements that are relevant for a clear understanding of the invention, while eliminating, for purposes of clarity, other elements that those of ordinary skill in the art will appreciate may also comprise a portion of the invention. However, because such elements are well known in the art, and because they do not necessarily facilitate a better understanding of the invention, a description of such elements is not provided herein.

Terms defined in the present disclosure are only used for describing specific embodiments of the present disclosure and are not intended to limit the scope of the present disclosure. Terms provided in singular forms are intended to include plural forms as well, unless the context clearly indicates otherwise. All of the terms used herein, including technical or scientific terms, have the same meanings as those generally understood by an ordinary person skilled in the related art, unless otherwise defined herein. Terms defined in a generally used dictionary should be interpreted as having meanings that are the same as or similar to the contextual meanings of the relevant technology and should not be interpreted as having ideal or exaggerated meanings, unless expressly so defined herein. In some cases, terms defined in the present disclosure should not be interpreted to exclude the embodiments of the present disclosure.

Provided herein are imaging systems for a patient comprising an imaging probe and an imaging assembly. The imaging probe comprises an elongate shaft comprising a proximal end, a distal portion, and a lumen extending between the proximal end and the distal portion; a rotatable optical core comprising a proximal end and a distal end, wherein at least a portion of the rotatable optical core is positioned within the lumen of the elongate shaft; and an optical assembly positioned proximate the distal end of the rotatable optical core, the optical assembly configured to direct light to tissue and collect reflected light from the tissue.

The imaging systems of the present inventive concepts can be used to provide image data representing arteries, veins, and/or other body conduits, and to image one or more devices inserted into those conduits. The imaging systems can provide image data related to healthy tissue, as well as diseased tissue, such as blood vessels including a stenosis, lesion, myocardial bridge, and/or other vessel narrowing (“stenosis” herein) and/or blood vessels including an aneurysm. The system can be configured to provide treatment information, wherein the treatment information is used by an operator to plan a treatment and/or predict a treatment outcome.

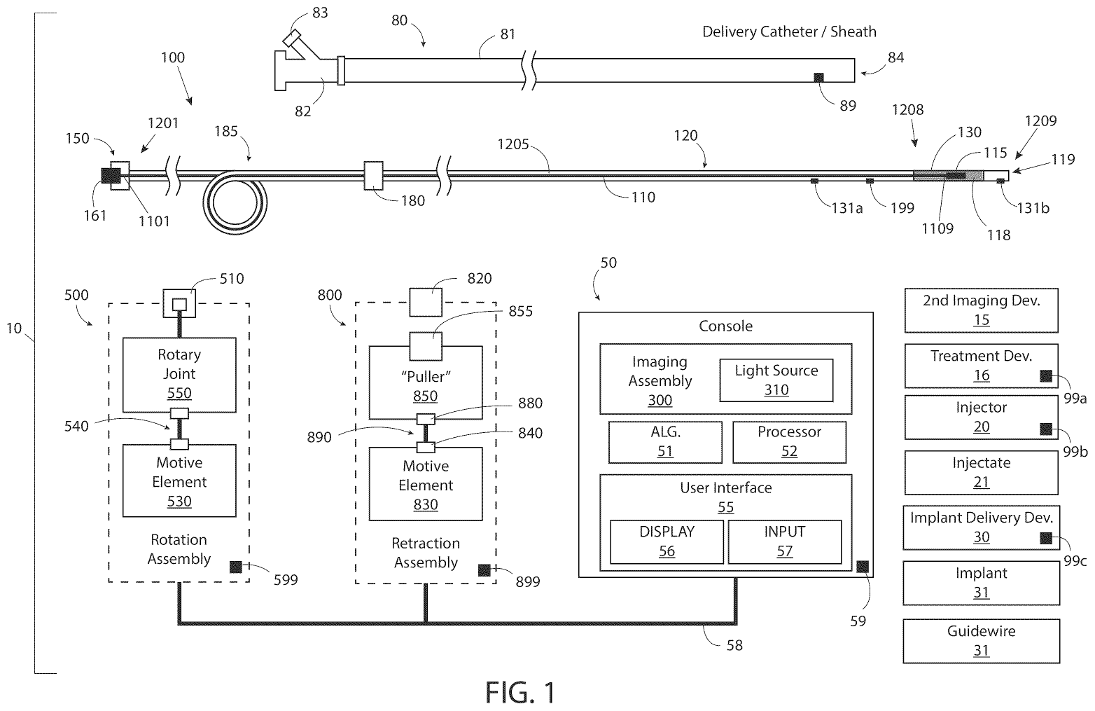

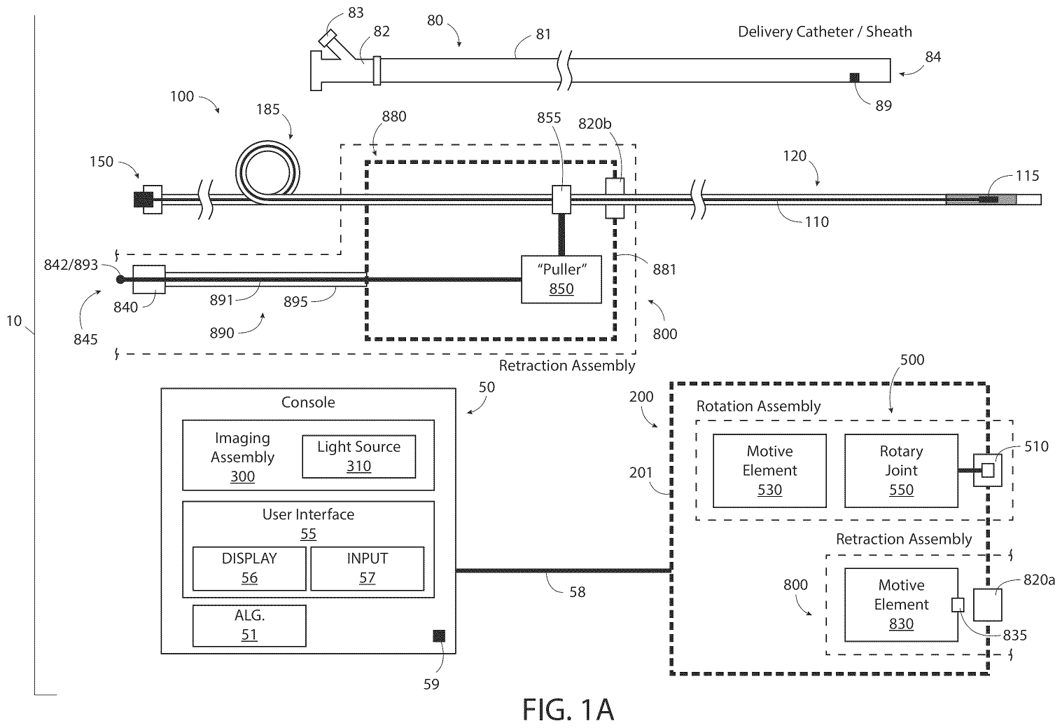

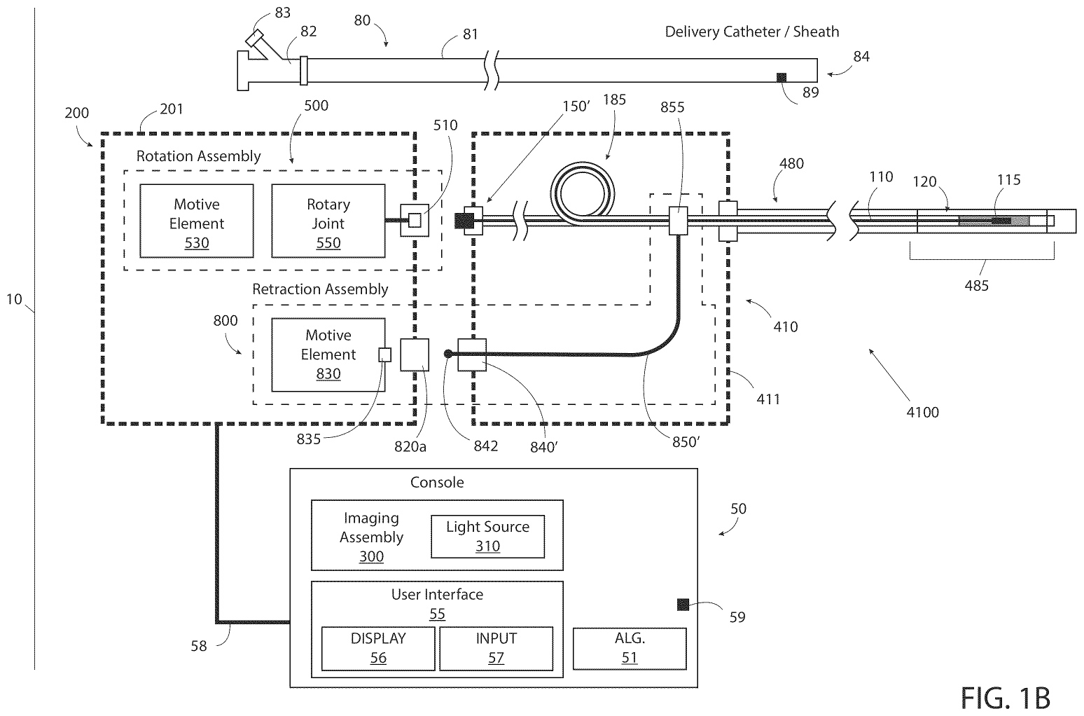

Referring now to FIG. 1, a schematic view of an imaging system comprising an imaging probe and independent retraction and rotation assemblies is illustrated, consistent with the present inventive concepts. Imaging system 10 is constructed and arranged to collect image data and produce one or more images based on the recorded data, such as when imaging system 10 comprises an Optical Coherence Tomography (OCT) imaging system constructed and arranged to collect image data of an imaging location (e.g. a segment of a blood vessel, such as during a pullback procedure). Imaging system 10 comprises a catheter-based probe, imaging probe 100, as well as a rotation assembly 500 and a retraction assembly 800, each of which can operably attach to imaging probe 100. Imaging system 10 can further comprise console 50 which is configured to operably connect to imaging probe 100, such as via rotation assembly 500 and/or retraction assembly 800. Imaging probe 100 can be introduced into a conduit of the patient, such as a blood vessel or other conduit of the patient, using one or more delivery catheters, for example delivery catheter 80 shown. Additionally or alternatively, imaging probe 100 can be introduced through an introducer device, such as an endoscope, arthroscope, balloon dilator, or the like. In some embodiments, imaging probe 100 is configured to be introduced into a conduit selected from the group consisting of: an artery; a vein; an artery within or proximate the heart; a vein within or proximate the heart; an artery within or proximate the brain; a vein within or proximate the brain; a peripheral artery; a peripheral vein; through a natural body orifice into a conduit, such as the esophagus; through a surgically created orifice into a body cavity, such as the abdomen; and combinations of one or more of these. Imaging system 10 can further comprise multiple imaging devices, second imaging device 15 shown. Imaging system 10 can further comprise a device configured to treat the patient, treatment device 16. Imaging system 10 can further comprise a fluid injector, such as injector 20, which can be configured to inject one or more fluids, such as a flushing fluid, an imaging contrast agent (e.g. a radiopaque contrast agent, hereinafter “contrast”) and/or other fluid, such as injectate 21 shown. Imaging system 10 can further comprise an implant, such as implant 31, which can be implanted in the patient via a delivery device, such as an implant delivery device 30 and/or delivery catheter 80.

In some embodiments, imaging probe 100 and/or another component of imaging system 10 can be of similar construction and arrangement to the similar components described in applicants co-pending U.S. patent application Ser. No. 15/566,041, titled “Micro-Optic Probes for Neurology”, filed Oct. 12, 2017; the content of which is incorporated herein by reference in its entirety for all purposes. Imaging probe 100 can be constructed and arranged to collect image data from a patient site, such as an intravascular cardiac site, an intracranial site, or other site accessible via the vasculature of the patient. In some embodiments, imaging system 10 can be of similar construction and arrangement to the similar systems and their methods of use described in applicants co-pending U.S. patent application Ser. No. 15/751,570, titled “Imaging System Includes Imaging Probe and Delivery Devices”, filed Feb. 9, 2018; the content of which is incorporated herein by reference in its entirety for all purposes.

Delivery catheter 80 comprises an elongate shaft, shaft 81, with a lumen 84 therethrough, and a connector 82 positioned on its proximal end. Connector 82 can comprise a Touhy or valved connector, such as a valved connector configured to prevent fluid egress from the associated delivery catheter 80 (with and/or without a separate shaft positioned within the connector 82). Connector 82 can comprise a port 83, such as a port constructed and arranged to allow introduction of fluid into delivery catheter 80 and/or for removing fluids from delivery catheter 80. In some embodiments, a flushing fluid, as described herein, is introduced via one or more ports 83, such as to remove blood or other undesired material from locations proximate optical assembly 115 (e.g. from a location proximal to optical assembly 115 to a location distal to optical assembly 115). Port 83 can be positioned on a side of connector 82 and can include a luer fitting and a cap and/or valve. Shafts 81, connectors 82, and ports 83 can each comprise standard materials and be of similar construction to commercially available introducers, guide catheters, diagnostic catheters, intermediate catheters and microcatheters used in interventional procedures. Delivery catheter 80 can comprise a catheter configured to deliver imaging probe 100 to an intracerebral location, an intracardiac location; and/or another location within a patient.

Imaging system 10 can comprise two or more delivery catheters 80, such as three or more delivery catheters 80. Multiple delivery catheters 80 can comprise at least a vascular introducer, and other delivery catheters 80 that can be inserted into the patient therethrough, after the vascular introducer is positioned through the skin of the patient. Two or more delivery catheters 80 can collectively comprise sets of inner diameters (IDs) and outer diameters (ODs) such that a first delivery catheter 80 slidingly receives a second delivery catheter 80 (e.g. the second delivery catheter OD is less than or equal to the first delivery catheter ID), and the second delivery catheter 80 slidingly receives a third delivery catheter 80 (e.g. the third delivery catheter OD is less than or equal to the second delivery catheter ID), and so on. In these configurations, the first delivery catheter 80 can be advanced to a first anatomical location, the second delivery catheter 80 can be advanced through the first delivery catheter to a second anatomical location distal or otherwise remote (hereinafter “distal”) to the first anatomical location, and so on as appropriate, using sequentially smaller diameter delivery catheters 80. In some embodiments, delivery catheters 80 can be of similar construction and arrangement to the similar components described in applicants co-pending U.S. patent application Ser. No. 15/751,570, titled “Imaging System Includes Imaging Probe and Delivery Devices”, filed Feb. 9, 2018; the content of which is incorporated herein by reference in its entirety for all purposes.

Imaging probe 100 comprises an elongate body, comprising one or more elongate shafts and/or tubes, elongate shaft 120 herein. Shaft 120 comprises a proximal end 1201, distal end 1209, and a lumen 1205 extending therebetween. In some embodiments, lumen 1205 can include multiple coaxial lumens within the one or more elongate shafts 120, such as one or more lumens abutting each other to define a single lumen 1205. In some embodiments, at least a portion of shaft 120 comprises a torque shaft. In some embodiments,

Attorney Docket No. SPR-013-US-CONI a portion of shaft 120 comprises a braided construction. In some embodiments, a portion of shaft 120 comprises a spiral cut tube (e.g. a spiral cut metal tube). In some embodiments, the pitch of the spiral cut can be varied along the length of the cut, such as to vary the stiffness of shaft 120 along the cut. A portion of shaft 120 can comprise a tube constructed of nickel-titanium alloy. Shaft 120 operably surrounds a rotatable optical fiber, optical core 110 (e.g. optical core 110 is positioned within lumen 1205), comprising a proximal end 1101 and a distal end 1109. Optical core 110 can comprise a dispersion shifted optical fiber, such as a depressed cladding dispersion shifted fiber (e.g. a Non-Zero Dispersion Shifted, NZDS, fiber). Shaft 120 further comprises a distal portion 1208, including a transparent window, window 130 (e.g. a window that is relatively transparent to the one or more frequencies of light transmitted through optical core 110). An optical assembly, optical assembly 115, is operably attached to the distal end 1109 of optical core 110. Optical assembly 115 is positioned within window 130 of shaft 120. Optical assembly 115 can comprise a GRIN lens optically coupled to the distal end 1109 of optical core 110. Optical assembly 115 can comprise a construction and arrangement similar to optical assembly 115 as described in applicant's co-pending International PCT Patent Application Serial Number PCT/US2018/062766, titled “Imaging System”, filed Nov. 28, 2018, and applicant's co-pending International PCT Patent Application Serial Number PCT/US2019/051447, titled “Imaging System with Optical Pathway”, filed Sep. 17, 2019, the content of each of which is incorporated herein by reference in its entirety for all purposes. A connector assembly, connector assembly 150, is positioned on the proximal end of shaft 120. Connector assembly 150 operably attaches imaging probe 100 to rotation assembly 500, as described herein. Connector assembly 150 surrounds and operably attaches to an optical connector 161, fixedly attached to the proximal end of optical core 110. A second connector, pullback connector 180, is positioned on shaft 120. Connector 180 can be removably attached and/or adjustably positioned along the length of shaft 120. Connector 180 can be positioned along shaft 120, such as by a clinician or other user of system 10, proximate the proximal end of delivery catheter 80 after imaging probe 100 has been inserted into a patient via delivery catheter 80. Shaft 120 can comprise a portion between connector assembly 150 and the placement location of connector 180 that accommodates slack in shaft 120, a proximal portion of shaft 120 (e.g. a proximal portion of imaging probe 100), service loop 185.

Imaging probe 100 can comprise one or more visualizable markers along its length (e.g. along shaft 120), markers 131a-b shown (marker 131 herein). Marker 131 can comprise markers selected from the group consisting of: radiopaque markers; ultrasonically reflective markers; magnetic markers; ferrous material; and combinations of one or more of these. In some embodiments, marker 131 comprises a marker positioned at a location (e.g. a location within and/or at least proximate distal portion 1208) to assist a user of imaging system 10 in performing a pullback procedure (“pullback procedure” or “pullback” herein), such as to cause tip 119 to be positioned at a location distal to the proximal end of an implant after the pullback is completed (e.g. so that imaging probe 100 can be safely advanced through the implant after the pullback).

In some embodiments, imaging probe 100 includes a viscous dampening material, gel 118, positioned within shaft 120 and surrounding optical assembly 115 and a distal portion of optical core 110 (e.g. a gel injected or otherwise installed in a manufacturing process). Gel 118 can comprise a non-Newtonian fluid, for example a shear-thinning fluid. In some embodiments, gel 118 comprises a static viscosity of greater than 500 centipoise, and a shear viscosity that is less than the static viscosity. In these embodiments, the ratio of static viscosity to shear viscosity of gel 118 can be between 1.2:1 and 100:1. Gel 118 can comprise a gel as described in reference to applicants co-pending U.S. patent application Ser. No. 15/566,041, titled “Micro-Optic Probes for Neurology”, filed Oct. 12, 2017, and applicant's co-pending International PCT Patent Application Serial Number PCT/US2018/062766, titled “Imaging System”, filed Nov. 28, 2018, the content of each of which is incorporated herein by reference in its entirety for all purposes.

Imaging probe 100 can include a distal tip portion, distal tip 119. In some embodiments, distal tip 119 can comprise a spring tip, such as a spring tip configured to improve the “navigability” of imaging probe 100 (e.g. to improve “trackability” and/or “steerability” of imaging probe 100), for example within a tortuous pathway (e.g. within a blood vessel of the brain or heart with a tortuous pathway). In some embodiments, tip 119 comprises a length of between 5 mm and 100 mm (e.g. a spring with a length between 5 mm and 100 mm). In some embodiments, spring tip 119 can comprise a user shapeable spring tip (e.g. at least a portion of spring tip 119 is malleable). Imaging probe 100 can be rotated (e.g. via connector 180) to adjust the direction of a non-linear shaped portion of spring tip 119 (e.g. to adjust the trajectory of spring tip 119 in the vasculature of the patient). Alternatively or additionally, tip 119 can comprise a cap, plug, or other element configured to seal the distal opening of window 130. In some embodiments, tip 119 can comprise a radiopaque marker configured to increase the visibility of imaging probe 100 under an X-ray or fluoroscope. In some embodiments, tip 119 can comprise a relatively short luminal guidewire pathway to allow “rapid exchange” translation of imaging probe 100 over a guidewire of system 10 (such as guidewire 31).

In some embodiments, at least the distal portion of imaging probe 100 (e.g. the distal portion of shaft 120 surrounding optical assembly 115) comprises an outer diameter of no more than 0.030″, such as no more than 0.025″, no more than 0.020″, and/or no more than 0.016″.

In some embodiments, imaging probe 100 can be constructed and arranged for use in an intravascular neural procedure (e.g. a procedure in which the blood, vasculature, and other tissue proximate the brain are visualized, and/or devices positioned temporarily or permanently proximate the brain are visualized). An imaging probe 100 configured for use in a neural procedure can comprise an overall length of at least 150 cm, such as a length of approximately 300 cm.

Alternatively or additionally, imaging probe 100 can be constructed and arranged for use in an intravascular cardiac procedure (e.g. a procedure in which the blood, vasculature, and other tissue proximate the heart are visualized, and/or devices positioned temporarily or permanently proximate the heart are visualized). An imaging probe 100 configured for use in a cardiovascular procedure can comprise an overall length of at least 120 cm, such as an overall length of approximately 280 cm (e.g. to allow placement of the proximal end of probe 100 outside of the sterile field). In some embodiments, such as for placement outside of the sterile field, imaging probe 100 can comprise a length greater than 220 cm and/or less than 320 cm.

Rotation assembly 500 comprises a connector assembly 510, operably attached to a rotary joint 550. Rotation assembly 500 further comprises a motor or other rotational energy source, motive element 530. Motive element 530 is operably attached to rotary joint 550 via a linkage assembly 540. In some embodiments, linkage assembly 540 comprises one or more gears, belts, pulleys, or other force transfer mechanisms. Motive element 530 can drive (e.g. rotate via linkage assembly 540) rotary joint 550 (and in turn core 110) at speeds of at least 100 rotations per second, such as at least 200 rotations per second, 250 rotations per second, 400 rotations per second, 500 rotations per second, or between 20 rotations per second and 1000 rotations per second. Motive element 530 can comprise a mechanism selected from the group consisting of: a motor; a servo; a stepper motor (e.g. a stepper motor including a gear box); a linear actuator; a hollow core motor; and combinations thereof. In some embodiments, rotation assembly 500 is configured to rotate optical assembly 115 and rotatable optical core 110 in unison.

Connector assembly 510 operably attaches to connector assembly 150 of imaging probe 100, allowing optical connector 161 to operably engage rotary joint 550. In some embodiments, connector assembly 510 operably engages connector assembly 150. In some embodiments, connector assembly 510 operably engages connector assembly 150 such that rotary joint 550 and optical connector 161 are free to rotate within the engaged assemblies.

Retraction assembly 800 comprises a connector assembly 820, that operably attaches to a reference point, for example connector 82 of delivery catheter 80, such as to establish a reference for retraction assembly 800 relative to the patient. Connector assembly 820 can attach to a reference point such as a patient introduction device, surgical table, and/or another fixed or semi fixed point of reference. A retraction element, puller 850, releasably attaches to connector 180 of imaging probe 100, such as via a carrier 855. Retraction assembly 800 retracts at least a portion of imaging probe 100 (e.g. the portion of imaging probe 100 distal to the attached connector 180), relative to the established reference. In some embodiments, retraction assembly 800 is configured to retract at least a portion of imaging probe 100 (e.g. at least optical assembly 115 and a portion of shaft 120) at a rate of between 5 mm/sec and 100 mm/sec, such as 60 mm/sec. In some embodiments, retraction assembly 800 is configured to retract at least a portion of imaging probe 100 at a rate of at least 60 mm/sec, at least 80 mm/sec, at least 100 mm/sec, and/or at least 150 mm/sec. Additionally or alternatively, the pullback procedure can be performed during a time period of between 0.5 sec and 25 sec, for example approximately 20 sec (e.g. over a distance of 100 mm at 5 mm/sec). Service loop 185 of imaging probe 100 can be positioned between retraction assembly 800 and/or at least connector assembly 820, and rotation assembly 500, such that imaging probe 100 can be retracted relative to the patient while rotation assembly 500 remains stationary (e.g. attached to the surgical table and/or to a portion of console 50).

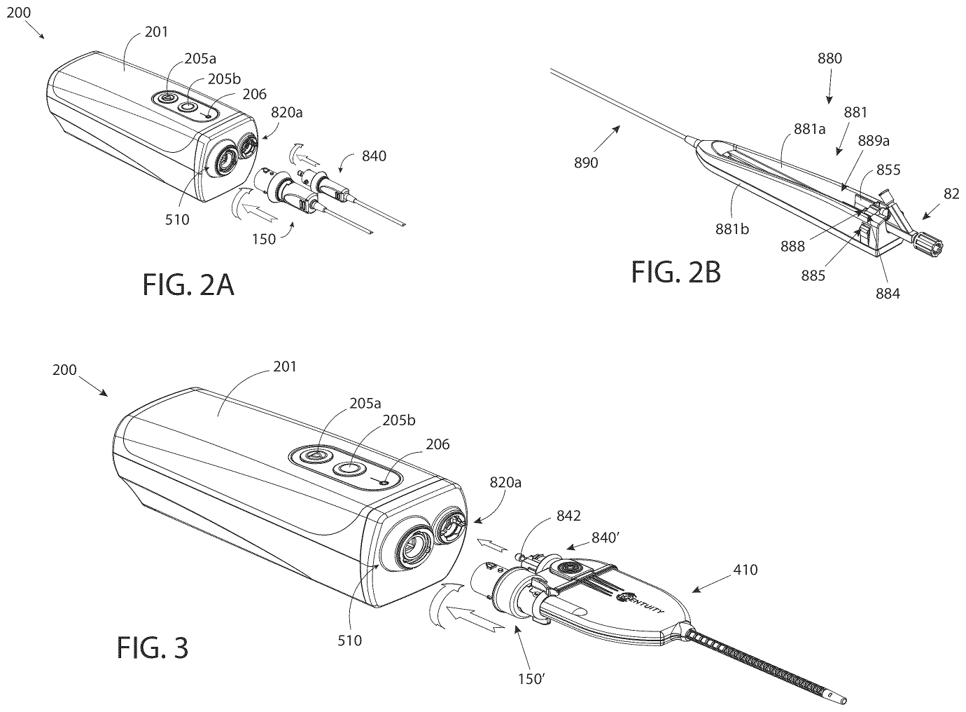

Retraction assembly 800 further comprises a linear drive, motive element 830. In some embodiments, motive element 830 can comprise a linear actuator, a worm drive operably attached to a motor, a pulley system, and/or other linear force transfer mechanisms. Puller 850 can be operably attached to motive element 830 via a linkage assembly 890. In some embodiments, linkage assembly 890 can comprise one or more components of a “pullback assembly”, as described in reference to FIGS. 1A and 2A. Alternatively or additionally, linkage assembly 890 can comprise one or more components of an enclosed pullback connector, as described in reference to FIG. 1B. One or more components of linkage assembly 890 can establish a frame of reference (e.g. an internal pullback reference) between puller 850 and the motive element 830, such that motive element 830 applies a pullback force to puller 850 via linkage assembly 890, and puller 850 retracts relative to the distal portion of linkage assembly 890 (e.g. relative to the distal end of sheath 895 as described in reference to FIG. 1A). In some embodiments, the distal end of linkage assembly 890 and connector assembly 820 are fixed relative to each other, and puller 850 translates linearly between the two in reaction to a force applied from motive element 830.

Console 50 comprises an imaging assembly 300, a user interface 55, processor 52, and one or more algorithms 51. Processor 52 can include one or more memory storage components, such as one or more memory circuits which store software routines, algorithms (e.g. algorithm 51), and other operating instructions of system 10, as well as data acquired by imaging probe 100, second imaging device 15, and/or another component of system 10. Imaging assembly 300 can be configured to provide light to optical assembly 115 (e.g. via optical core 110) and collect light from optical assembly 115 (e.g. via optical core 110). Imaging assembly 300 can include a light source 310. Light source 310 can comprise one or more light sources, such as one or more light sources configured to provide one or more wavelengths of light to optical assembly 115 via optical core 110. Light source 310 is configured to provide light to optical assembly 115 (via optical core 110) such that image data can be collected comprising cross-sectional, longitudinal and/or volumetric information related to a patient site or implanted device being imaged. Light source 310 can be configured to provide light such that the image data collected includes characteristics of tissue within the patient site being imaged, such as to quantify, qualify or otherwise provide information related to a patient disease or disorder present within the patient site being imaged. Light source 310 can be configured to deliver broadband light and have a center wavelength in the range from 350 nm to 2500 nm, from 800 nm to 1700 nm, from 1280 nm to 1310 nm, or approximately 1300 nm (e.g. light delivered with a sweep range from 1250 nm to 1350 nm). Light source 310 can comprise a sweep rate of at least 50 KHz. In some embodiments, light source 310 comprises a sweep rate of at least 100 KHz, such as at least 200 Khz, 300 KHz, 400 KHz, and/or 500 KHz. These faster sweep rates provide numerous advantages, such as to provide a higher frame rate, as well as being compatible with rapid pullback and rotation rates. For example, the higher sweep rate enables the requisite sampling density (e.g. the amount of luminal surface area swept by the rotating beam) to be achieved in a shorter time, advantageous in most situations and especially advantageous when there is relative motion between the probe and the surface/tissue being imaged such as arteries in a beating heart. Light source 310 bandwidth can be selected to achieve a desired resolution, which can vary according to the needs of the intended use of imaging system 10. In some embodiments, bandwidths are about 5% to 15% of the center wavelength, which allows resolutions of between 20 μm and 5 μm. Light source 310 can be configured to deliver light at a power level meeting ANSI Class 1 (“eye safe”) limits, though higher power levels can be employed. In some embodiments, light source 310 delivers light in the 1.3 μm band at a power level of approximately 20 mW. Tissue light scattering is reduced as the center wavelength of delivered light increases, however water absorption increases. Light source 310 can deliver light at a wavelength approximating 1300 nm to balance these two effects. Light source 310 can be configured to deliver shorter wavelength light (e.g. approximately 800 nm light) to traverse patient sites to be imaged including large amounts of fluid. Alternatively or additionally, light source 310 can be configured to deliver longer wavelengths of light (e.g. approximately 1700 nm light), such as to reduce a high level of scattering within a patient site to be imaged. In some embodiments, light source 310 comprises a tunable light source (e.g. light source 310 emits a single wavelength that changes repetitively over time), and/or a broad-band light source. Light source 310 can comprise a single spatial mode light source or a multimode light source (e.g. a multimode light source with spatial filtering).

Light source 310 can comprise a relatively long effective coherence length, such as a coherence length of greater than 10 mm, such as a length of at least 50 mm, at all frequencies within the bandwidth of the light source. This coherence length capability enables longer effective scan ranges to be achieved by system 10, as the light returning from distant objects to be imaged (e.g. tissue) must remain in phase coherence with the returning reference light, in order to produce detectable interference fringes. In the case of a swept-source laser, the instantaneous linewidth is very narrow (i.e. as the laser is sweeping, it is outputting a very narrow frequency band that changes at the sweep rate). Similarly, in the case of a broad-bandwidth source, the detector arrangement must be able to select very narrow linewidths from the spectrum of the source. The coherence length scales inversely with the linewidth. Longer scan ranges enable larger or more distant objects to be imaged (e.g. more distal tissue to be imaged). Current systems have lower coherence length, which correlates to reduced image capture range as well as artifacts (ghosts) that arise from objects outside the effective scan range.

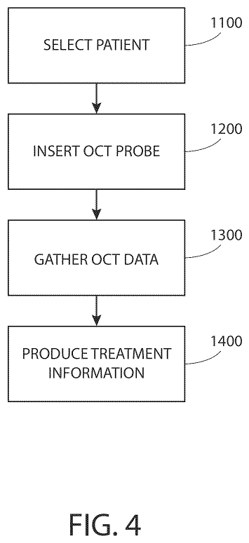

Console 50 can comprise one or more algorithms, such as algorithm 51 shown, which can be configured to adjust (e.g. automatically and/or semi-automatically adjust) one or more operational parameters of imaging system 10, such as an operational parameter of console 50, imaging probe 100 and/or a delivery catheter 80. Console 50 can further comprise a processing assembly, processor 52, configured to execute algorithm 51, and/or perform any type of data processing, such as digital signal processing, described in reference to FIG. 4. Additionally or alternatively, algorithm 51 can be configured to adjust an operational parameter of a separate device, such as injector 20 or implant delivery device 30 described herein. In some embodiments, algorithm 51 is configured to adjust an operational parameter based on one or more sensor signals, such as a sensor signal provided by a sensor-based functional element of the present inventive concepts as described herein. Algorithm 51 can be configured to adjust an operational parameter selected from the group consisting of: a rotational parameter such as rotational velocity of optical core 110 and/or optical assembly 115; a retraction parameter of shaft 120 and/or optical assembly 115 such as retraction velocity, distance, start position, end position and/or retraction initiation timing (e.g. when retraction is initiated); a position parameter such as position of optical assembly 115; a line spacing parameter such as lines per frame; an image display parameter such as a scaling of display size to vessel diameter; an imaging probe 100 configuration parameter; an injectate 21 parameter such as a saline to contrast ratio configured to determine an appropriate index of refraction; a light source 310 parameter such as power delivered and/or frequency of light delivered; and combinations of one or more of these. In some embodiments, algorithm 51 is configured to adjust a retraction parameter such as a parameter triggering the initiation of the pullback, such as a pullback that is initiated based on a parameter selected from the group consisting of: lumen flushing (the lumen proximate optical assembly 115 has been sufficiently cleared of blood or other matter that would interfere with image creation); an indicator signal is received from injector 20 (e.g. a signal indicating sufficient flushing fluid has been delivered); a change in image data collected (e.g. a change in an image is detected, based on the image data collected, that correlates to proper evacuation of blood from around optical assembly 115); and combinations of one or more of these. In some embodiments, algorithm 51 is configured to adjust an imaging system 10 configuration parameter related to imaging probe 100, such as when algorithm 51 identifies (e.g. automatically identifies via an RF or other embedded ID) the attached imaging probe 100 and adjusts an imaging system 10 parameter, such as an arm path length parameter, a dispersion parameter, and/or other parameter as listed above.

Imaging system 10 can comprise one or more interconnect cables, bus 58 shown. Bus 58 can operably connect rotation assembly 500 to console 50, retraction assembly 800 to console 50, and or rotation assembly 500 to retraction assembly 800. Bus 58 can comprise one or more optical transmission fibers, electrical transmission cables, fluid conduits, and combinations of one or more of these. In some embodiments, bus 58 comprises at least an optical transmission fiber that optically couples rotary joint 550 to imaging assembly 300 of console 50. Additionally or alternatively, bus 58 comprises at least power and/or data transmission cables that transfer power and/or motive information to one or more of motive elements 530 and 830.

Second imaging device 15 can comprise an imaging device such as one or more imaging devices selected from the group consisting of: an X-ray; a fluoroscope such as a single plane or biplane fluoroscope; a CT Scanner; an MRI; a PET Scanner; an ultrasound imager; and combinations of one or more of these. In some embodiments, second imaging device 15 comprises a device configured to perform rotational angiography.

Treatment device 16 can comprise an occlusion treatment or other treatment device selected from the group consisting of: a balloon catheter constructed and arranged to dilate a stenosis or other narrowing of a blood vessel; a drug eluting balloon; an aspiration catheter; a sonolysis device; an atherectomy device; a thrombus removal device such as a stent retriever device; a Trevo™ stentriever; a Solitaire™ stentriever; a Revive™ stentriever; an Eric™ stentriever; a Lazarus™ stentriever; a stent delivery catheter; a microbraid implant; an embolization system; a WEB™ embolization system; a Luna™ embolization system; a Medina™ embolization system; and combinations of one or more of these. In some embodiments, imaging probe 100 is configured to collect data related to treatment device 16 (e.g. treatment device 16 location, orientation and/or other configuration data), after treatment device 16 has been inserted into the patient.

Injector 20 can comprise a power injector, syringe pump, peristaltic pump or other fluid delivery device configured to inject a contrast agent, such as radiopaque contrast, and/or other fluids. In some embodiments, injector 20 is configured to deliver contrast and/or other fluid (e.g. contrast, saline and/or Dextran). In some embodiments, injector 20 delivers fluid in a flushing procedure as described herein. In some embodiments, injector 20 delivers contrast or other fluid through a delivery catheter 80 with an ID of between 5 Fr and 9 Fr, a delivery catheter 80 with an ID of between 0.53″ to 0.70″, or a delivery catheter 80 with an ID between 0.0165″ and 0.027″. In some embodiments, contrast or other fluid is delivered through a delivery catheter as small as 4 Fr (e.g. for distal injections). In some embodiments, injector 20 delivers contrast and/or other fluid through the lumen of one or more delivery catheters 80, while one or more smaller delivery catheters 80 also reside within the lumen. In some embodiments, injector 20 is configured to deliver two dissimilar fluids simultaneously and/or sequentially, such as a first fluid delivered from a first reservoir and comprising a first concentration of contrast, and a second fluid from a second reservoir and comprising less or no contrast.

Injectate 21 can comprise fluid selected from the group consisting of: optically transparent material; saline; visualizable material; contrast; Dextran; an ultrasonically reflective material; a magnetic material; and combinations thereof. Injectate 21 can comprise contrast and saline. Injectate 21 can comprise at least 20% contrast. During collection of image data, a flushing procedure can be performed, such as by delivering one or more fluids, injectate 21 (e.g. as propelled by injector 20 or other fluid delivery device), to remove blood or other somewhat opaque material (hereinafter non-transparent material) proximate optical assembly 115 (e.g. to remove non-transparent material between optical assembly 115 and a delivery catheter and/or non-transparent material between optical assembly 115 and a vessel wall), such as to allow light distributed from optical assembly 115 to reach and reflectively return from all tissue and other objects to be imaged. In these flushing embodiments, injectate 21 can comprise an optically transparent material, such as saline. Injectate 21 can comprise one or more visualizable materials, as described herein.

As an alternative or in addition to its use in a flushing procedure, injectate 21 can comprise material configured to be viewed by second imaging device 15, such as when injectate 21 comprises a contrast material configured to be viewed by a second imaging device 15 comprising a fluoroscope or other X-ray device; an ultrasonically reflective material configured to be viewed by a second imaging device 15 comprising an ultrasound imager; and/or a magnetic material configured to be viewed by a second imaging device 15 comprising an MRI.

Implant 31 can comprise an implant (e.g. a temporary or chronic implant) for treating one or more of a vascular occlusion or an aneurysm. In some embodiments, implant 31 comprises one or more implants selected from the group consisting of: a flow diverter; a Pipeline™ flow diverter; a Surpass™ flow diverter; an embolization coil; a stent; a Wingspan™ stent; a covered stent; an aneurysm treatment implant; and combinations of one or more of these.

Implant delivery device 30 can comprise a catheter or other tool used to deliver implant 31, such as when implant 31 comprises a self-expanding or balloon expandable portion. In some embodiments, imaging system 10 comprises imaging probe 100, one or more implants 31 and/or one or more implant delivery devices 30. In some embodiments, imaging probe 100 is configured to collect data related to implant 31 and/or implant delivery device 30 (e.g. implant 31 and/or implant delivery device 30 anatomical location, orientation and/or other configuration data), after implant 31 and/or implant delivery device 30 has been inserted into the patient.

In some embodiments, one or more system components, such as console 50, delivery catheter 80, imaging probe 100, rotation assembly 500, retraction assembly 800, treatment device 16, injector 20, and/or implant delivery device 30, further comprise one or more functional elements (“functional element” herein), such as functional elements 59, 89, 199, 599, 899, 99a, 99b, and/or 99c, respectively, shown. Each functional element can comprise at least two functional elements. Each functional element can comprise one or more elements selected from the group consisting of: sensor; transducer; and combinations thereof. The functional element can comprise a sensor configured to produce a signal. The functional element can comprise a sensor selected from the group consisting of: a physiologic sensor; a pressure sensor; a strain gauge; a position sensor; a GPS sensor; an accelerometer; a temperature sensor; a magnetic sensor; a chemical sensor; a biochemical sensor; a protein sensor; a flow sensor such as an ultrasonic flow sensor; a gas detecting sensor such as an ultrasonic bubble detector; a sound sensor such as an ultrasound sensor; and combinations thereof. The sensor can comprise a physiologic sensor selected from the group consisting of: a pressure sensor such as a blood pressure sensor; a blood gas sensor; a flow sensor such as a blood flow sensor; a temperature sensor such as a blood or other tissue temperature sensor; and combinations thereof. The sensor can comprise a position sensor configured to produce a signal related to a vessel path geometry (e.g. a 2D or 3D vessel path geometry). The sensor can comprise a magnetic sensor. The sensor can comprise a flow sensor. The system can further comprise an algorithm configured to process the signal produced by the sensor-based functional element. Each functional element can comprise one or more transducers. Each functional element can comprise one or more transducers selected from the group consisting of: a heating element such as a heating element configured to deliver sufficient heat to ablate tissue; a cooling element such as a cooling element configured to deliver cryogenic energy to ablate tissue; a sound transducer such as an ultrasound transducer; a vibrational transducer; and combinations thereof.

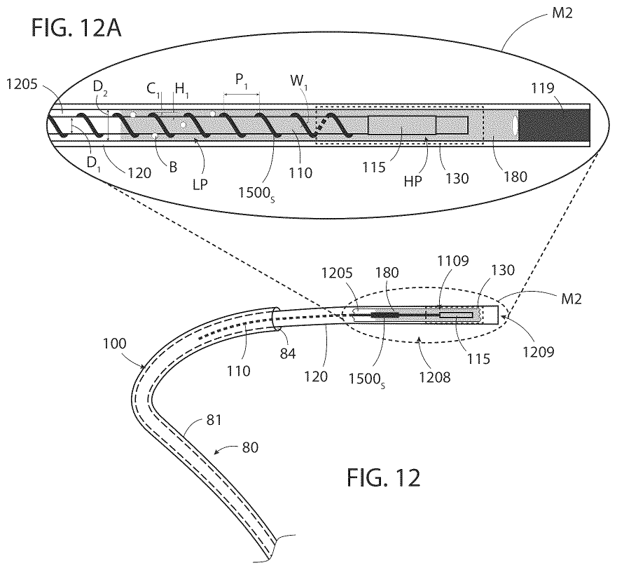

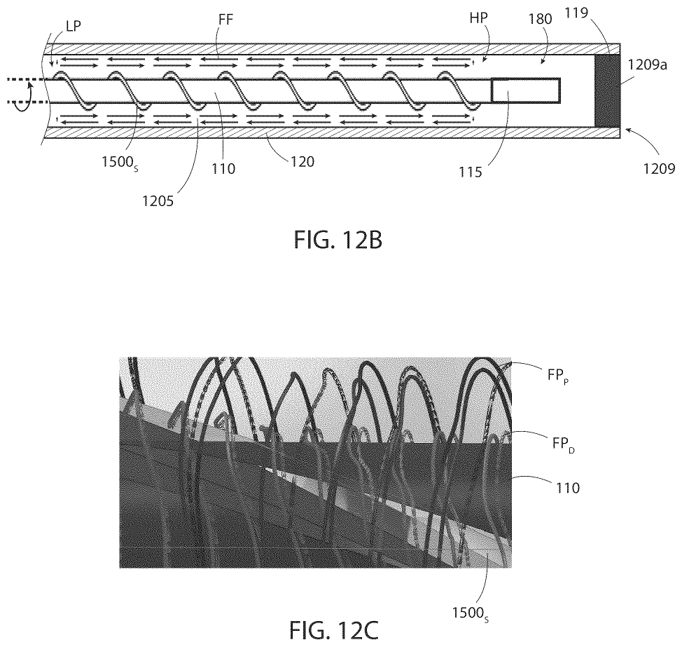

In some embodiments, imaging probe 100 comprises a fluid propulsion element and/or a fluid pressurization element (“fluid pressurization element” herein), not shown but such as FPE 1500s described in reference to FIGS. 12 and 12A-C. A fluid propulsion element can be configured to prevent and/or reduce the presence of bubbles within gel 118 proximate optical assembly 115. The fluid propulsion element can be fixedly attached to optical core 110, wherein rotation of optical core 110 in turn rotates the fluid propulsion element, such as to generate a pressure increase within gel 118 that is configured to reduce presences of bubbles from locations proximate optical assembly 115. Such one or more fluid pressurization elements can reduce the likelihood of bubble formation within gel 118, reduce the size of bubbles within gel 118, and/or move any bubbles formed within gel 118 away from a location that would adversely impact the collecting of image data by optical assembly 115 (e.g. move bubbles away from optical assembly 115). In some embodiments, a fluid propulsion element of imaging probe 100 comprises a similar construction and arrangement to a fluid propulsion element described in applicant's co-pending U.S. Provisional Patent Application Ser. No. 62/840,450, titled “Imaging Probe with Fluid Pressurization Element “, filed Apr. 30, 2019, the content of which is incorporated herein by reference in its entirety for all purposes.

Referring now to FIG. 1A, a schematic view of an imaging system is illustrated, the system comprising an imaging probe operably attachable to a patient interface module, and an independent pullback module operably attachable to the patient interface module and the imaging probe, consistent with the present inventive concepts. Imaging system 10 can comprise a patient interface module 200. Patient interface module 200 comprises a housing, housing 201, surrounding at least a portion of rotation assembly 500, and at least a portion of retraction assembly 800. Imaging system 10 can further comprise a second, discrete component, pullback module 880. Pullback module 880 comprises a housing, housing 881, surrounding at least a portion of retraction assembly 800. Pullback module 880 and patient interface module 200 can be operably attached to each other via a connector assembly, linkage assembly 890 described herein. Pullback module 880 and patient interface module 200 can be constructed and arranged (via each having a separate housing) to enable positioning at different locations (e.g. linkage assembly 890 connecting modules 880 and 200 can comprise a length of at least 15 cm such that the two remote locations can be at least 15 cm apart), for example patient interface module 200 can be positioned on or near a surgical bed rail, and pullback module 880 can be positioned near a vascular access site of the patient (e.g. within 30 cm of the vascular access site thru which imaging probe 100 enters the patient). Linkage assembly 890 can comprise a linkage 891 slidingly received within sheath 895. Linkage 891 is operably attached to puller 850, and the proximal end 893 of linkage 891 can comprise a connection point, 842. Motive element 830 can comprise a connector 835 configured to releasably attach to connection point 842. Components shown in FIG. 1A can be of similar construction and arrangement to like components described in reference to FIG. 1, and as described elsewhere herein.

Pullback module 880 can comprise a connector assembly 820b that operably attaches to connector 82 of delivery catheter 80, such as described in reference to FIG. 2B. Connector assembly 845 can comprise a connector 840 that operably attaches to a connector assembly 820a of patient interface module 200, as described in reference to FIG. 2A.

Referring now to FIG. 1B, a schematic view of an imaging system is illustrated, the system comprising an imaging probe operably attachable to a module comprising a first connector for attaching to a rotation motive element and a second connector for attaching to a retraction motive element, consistent with the present inventive concepts. Imaging system 10 can comprise a patient interface module 200 as described herein. Imaging system 10 can further comprise a connector module, module 410. Module 410 comprises a housing, housing 411, surrounding at least a portion of retraction assembly 800, service loop 185 of imaging probe 100, connector assembly 150′, and connector 840′. Module 410 can be configured to operably attach both imaging probe 100 and a linkage, puller 850′, to patient interface module 200. Components shown in FIG. 1B can be of similar construction and arrangement to like components described in reference to FIG. 1, and as described elsewhere herein. Module 410 can be operably attached to a delivery catheter 480. Delivery catheter 480 can be of similar construction and arrangement to delivery catheter 80 described in reference to FIG. 1. Delivery catheter 480 can comprise at least a portion that is optically transparent, window 485. Window 485 can be positioned at or near a distal portion of delivery catheter 480. Window 485 can comprise a material transparent to imaging modalities utilized by imaging probe 100, such that imaging probe 100 can image through window 485, for example when optical assembly 115 is retracted within window 485. In some embodiments, module 410, delivery catheter 480, and imaging probe 100 collectively form catheter assembly 4100.

Referring now to FIG. 2A, a perspective view of connectors being attached to a patient interface is illustrated, consistent with the present inventive concepts. Patient interface module 200 is configured to provide rotation to a rotatable optical core of an imaging probe, and to provide a motive force to translate at least a portion of the imaging probe, such as is described herein. Patient interface module 200 comprises rotation assembly 500, and at least a portion of retraction assembly 800. A housing 201 surrounds patient interface module 200. Patient interface module 200 can comprise one or more user interface elements, such as one or more inputs, buttons 205a,b, and one or more outputs, indicator 206 shown. Patient interface module 200 comprises a first physical connector assembly, connector assembly 510, for operably connecting to connector assembly 150 as described herein. Patient interface module 200 can further comprise a second physical connector assembly, connector assembly 820a, for operably connecting to connector 840 also as described herein. Connector assembly 150 and connector 840 can each comprise bayonet type connectors, constructed and arranged to be at least partially inserted into connector assemblies 510 and 820a, respectively. Connector assembly 150 and connector 840 can be subsequently rotated (e.g. an approximately 45° rotation) to lock their connections with connector assemblies 510 and 820a, respectively, as described herein. Connector assembly 150 and/or 840 can comprise numerous forms of connectors, such as a bayonet or other locking connectors.

Referring now to FIG. 2B, a perspective view of a pullback assembly is illustrated, consistent with the present inventive concepts. Pullback module 880 can be operably attached to a portion of an imaging probe 100 of the present inventive concepts, and provide a retraction force to the probe, pulling at least a portion of the probe proximally relative to a patient (e.g. relative to a patient introduction device), as described herein. Pullback module 880 can comprise a construction and arrangement similar to pullback module 880 as described in applicant's co-pending International PCT Patent Application Serial Number PCT/US2018/062766, titled “Imaging System”, filed Nov. 28, 2018, the content of which is incorporated herein by reference in its entirety. Pullback module 880 can be operably attached to the distal end of a linkage 891 (not shown). Linkage assembly 890 can be slidingly received through pullback module 880. Sheath 895 can be fixedly attached to the proximal end of module 880. Linkage 891 is slidingly received along the length of module 880 and is operably attached at its distal end to puller 850.

Pullback module 880 can comprise a two-part housing 881, including a top housing 881a and bottom housing 881b. Module 880 can contain a translating cart, puller 850 (not shown, but positioned below carrier 855, and as described herein). Puller 850 can be designed to translate within module 880. Module 880 can comprise a biasing element, spring 852 (not shown). Spring 852 can provide a biasing force to puller 850, such as to bias puller 850 distally.

Top housing 881a can comprise a first cavity, retention port 884 and a second cavity, trench 889. Retention port 884 and trench 889 can be separated by a projection, retention wall 888. Physical connector assembly 820b can comprise a retention port 884 of housing 881a, including wall 888, and a retention mechanism, clip 885. Clip 885 can be configured to releasably engage the proximal end of a delivery catheter such as sheath connector 82 of delivery catheter 80, such as when connector 82 comprises a Tuohy Borst connector. Physical connector assembly 820b can further comprise a biasing element, spring 886 (not shown). Spring 886 can provide a biasing force to maintain clip 885 in an engaged position about connector 82.

Pullback module 880 can further comprise a carrier 855. Carrier 855 can operably attach to puller 850, such as through a slot 889a in housing 881a. Carrier 855 can translate within trench 889 in response to puller 850, which translates in response to linkage 891. Carrier 855 can operably attach to a portion of imaging probe 100, such as to a pullback connector 180. Pullback connector 180 can comprise a “torquer”, or other device affixed to shaft 120 of imaging probe 100. Sheath 895 of linkage assembly 890 can provide a frame of reference between connector 840 and pullback module 880, such that when the proximal end of linkage 891 is retracted relative to connector 840, the distal end of linkage 891 is retracted towards sheath 895 (i.e. towards the proximal end of pullback module 880). This relative motion transfers motive force applied at connector 840 (e.g. via motive element 830, as described herein), to puller 850. Puller 850 subsequently transfers the motive force to imaging probe 100, and imaging probe 100 is retracted relative to the patient.

In operation, imaging probe 100 can be manually (e.g. by a user) advanced through the vasculature of the patient. Pullback module 880 can be attached to the patient (e.g. to delivery catheter 80 via connector 82, when delivery catheter 80 is inserted into and in a relatively fixed position relative to the patient), and connector 180 can be operably connected to imaging probe 100, and positioned proximate delivery catheter 80 (e.g. a torquer connector 180 can be tightened to imaging probe 100 proximate delivery catheter 80). Connector 180 (not shown) can be operably positioned within carrier 855, and a motive force can be applied to the distal end of linkage 891. Carrier 855 retracts within trench 889, retracting imaging probe 100 relative to the patient. After retraction, connector 180 can be removed from carrier 855 (e.g. lifted out of), and carrier 855 and imaging probe 100 can be re-advanced independently. For example, carrier 855 can re-advance via the bias of spring 852, as the proximal end of linkage 891 is allowed to advance, and imaging probe 100 can be re-advanced manually by a user. Subsequent retractions can be performed by repositioning connector 180 in carrier 855 after both have been re-advanced. Carrier 855 can comprise a capturing portion, such as a “cup-like” geometry, a hook, or other capture-enabling portion, such that carrier 855 can only impart a retraction force on connector 180. In this configuration, if carrier 855 were to translate distally, connector 180 would automatically disengage from carrier 855 (e.g. connector 180 would fall out of the cup portion of carrier 855).

Referring now to FIG. 3, a perspective view of connectors being attached to a patient interface module is illustrated, consistent with the present inventive concepts. Patient interface module 200 can be of similar construction and arrangement to patient interface module 200 as described in reference to FIG. 2A. Patient interface module 200 comprises a first physical connector assembly, connector assembly 510, for operably connecting to connector assembly 150′. Patient interface module 200 can further comprise a second physical connector assembly, connector assembly 820a, for operably connecting to connector 840′. Connector assembly 150′ and connector 840′ can each comprise bayonet type connectors, constructed and arranged to be at least partially inserted into connector assemblies 510 and 820a, respectively.

As described herein, system 10 can be constructed and arranged to provide improved imaging of a patient's anatomy (e.g. of one or more blood vessels of the patient) as well as improved imaging of implants, catheters, and/or other devices positioned in the patient (e.g. positioned in a blood vessel of the patient). In some embodiments, system 10 is configured to provide information that is used (e.g. by a clinician) to perform a treatment (e.g. an intervention), wherein the information is based on, at least, optical coherence tomography data. For example, OCT and other data gathered by system 10, can be used to plan a treatment and/or predict a treatment outcome (e.g. the planning and/or predicting performed by system 10, a user of system 10, or a combination of the two), such as to impact a treatment to be delivered to the patient (“OCT-guided treatment” and/or “OCT-guided therapy” herein).

As described herein, imaging probe 100 can comprise at least one of: size (e.g. diameter and/or length), scan range, flexibility, and/or imaging capability configured to provide the improved imaging. Imaging probe 100 can comprise a size and/or flexibility configured to enable imaging of tight lesions within the vessel. As used herein, a tight lesion can comprise a lesion whose resultant lumen (i.e. the lumen within the lesion) comprises a diameter (e.g. the smallest diameter along the length of the lesion) of less than 2 mm (0.080″). A commercially available OCT catheter positioned to image a lesion with a lumen of this small diameter would effectively block the proximally-applied flush media from propagating to locations distal to the lesion, preventing the use of this commercial device. However, imaging probe 100 can be constructed and arranged to image these tight lesions, for example lesions with a resultant lumen as small as 1.5 mm (0.060″), as low as 1.3 mm (0.053″), 1.1 mm (0.043″), and/or as low as 0.9 mm (0.036″) can be imaged by imaging probe 100. For example, the distal portion of imaging probe 100 can comprise an outer diameter of no more than 2.6 F (0.034″), such as an outer diameter of no more than 1.7 F (0.022″), such as to enable system 10 to be used to image potential vessels (e.g. arteries) to be treated that have a tight lesion, such as when the distal portion of imaging probe 100 is inserted into and through a stenosis, such as in a “pre-treatment” imaging procedure (e.g. a procedure performed prior to intervention or other treatment of the stenosis). As described herein, currently available OCT imaging systems can be too large to provide useful data (e.g. unable to pass thru and/or provide sufficient blood clearing in a tight lesion). Other types of imaging systems, such as angiography, may not provide sufficiently accurate results when imaging tight lesions (e.g. erroneously indicate no treatment is warranted, such as when providing FFR information). In some embodiments, system 10 is used to perform a pre-treatment imaging procedure (e.g. of a tight lesion) to gather data to enable OCT-guided treatment in which the data provided by system 10 (e.g. using images from at least probe 100) is used by an operator (e.g. a clinician) to make decisions about a future treatment to be performed. In these embodiments, system 10 can also be used to image a similar anatomical location, after the treatment has been performed (in a “post-treatment” imaging procedure).

In some embodiments, system 10 is configured to perform a pre-treatment imaging procedure (e.g. of a tight lesion or otherwise) and provide OCT-guided treatment due to the following characteristics of system 10: distal portion of probe 100 (e.g. including optical assembly 115) comprises a diameter of less than 2.6 Fr (0.034″), such as a diameter of no more than 2.0 Fr (0.026″), such as a diameter of no more than 1.7 Fr (0.022″)

In some embodiments, system 10 is configured to perform a pre-treatment imaging procedure (e.g. of a tight lesion or otherwise) and provide OCT-guided treatment due to the following characteristics of system 10: optical assembly 115 is rotated (e.g. via rotation assembly 500) at a rate of more than 180 rotations per second, such as a rate of at least 200, 250, 400, and/or 500 rotations per second.

In some embodiments, system 10 is configured to perform a pre-treatment imaging procedure (e.g. of a tight lesion or otherwise) and provide OCT-guided treatment due to the following characteristics of system 10: scan range of system 10 is at least a radius of 7 mm, such as a radius of at least 11 mm. The long scan range of system 10 provides numerous advantages, such as the ability to image from the imaged vessel into any side branches of that vessel, the ability to image large vessels when optical assembly 115 is eccentrically positioned within the vessel lumen (e.g. proximate a portion of the vessel wall), and/or the ability to image larger vessels in general, such as the left main artery, carotid arteries, and large peripheral arteries.

In some embodiments, system 10 is configured to perform a pre-treatment imaging procedure (e.g. of a tight lesion or otherwise) and provide OCT-guided treatment due to the following characteristics of system 10: pullback distance of more than 7.5 cm, such as a pullback of at least 10 cm, or at least 15 cm. The pullback can be performed at a rate of at least 25 mm/sec, and/or within a time period of no more than 4 seconds (e.g. a complete pullback of at least 7.5 cm, 10 cm, and/or 15 cm in no more than 4 seconds). The operable pullback speed of imaging probe 100 can be determined via a relationship between the rotation rate of optical assembly 115 and the desired frame density (e.g. frames/mm) of the OCT image data, such that the pullback speed comprises the rotation rate divided by the frame density. Imaging probe 100 can comprise a rotation rate of greater than 180 Hz, such as at least 200 Hz or at least 250 Hz. Imaging probe 100 can comprise a frame spacing of no more than 0.2 mm (i.e. a frame density of at least 5 frames/mm). Imaging probe 100 can comprise a laser scan frequency of at least 200 KHz.

In some embodiments, system 10 is configured to perform a pre-treatment imaging procedure (e.g. of a tight lesion or otherwise) and provide OCT-guided treatment due to the following characteristics of system 10: pullback speed (the translation rate of optical assembly 115 during a pullback) of at least 50 mm/s. In these embodiments, rotation rate of optical assembly 115 can be at least 180 Hz, 200 Hz, and/or 250 Hz. In these embodiments, the frame spacing can be 0.2 mm minimum.

In some embodiments, system 10 is configured to perform a pre-treatment imaging procedure (e.g. of a tight lesion or otherwise) and provide OCT-guided treatment due to the following characteristics of system 10: lines per frame of at least 400, such as at least 800 lines/frame, where a frame comprises approximately 360° of continuous image data (i.e. one full rotation of optical assembly 115 provides one frame of image data). In some embodiments, system 10 is configured to capture frames at a rate sufficient to allow down-sampling of the frames (e.g. down sampling performed prior to analog to digital conversion of the data, and/or other bandwidth-limited data processing).

In some embodiments, system 10 is configured to perform a pre-treatment imaging procedure (e.g. of a tight lesion or otherwise) and provide OCT-guided treatment due to the following characteristics of system 10: scan frequency of at least 50 kHz, such as at least 200 kHz, 350 kHz, and/or 500 kHz. In these embodiments, the lines per frame can be at least 400 lines/frame, or at least 800 lines/frame (e.g. where lines per frame equals the scan frequency divided by the rotation rate of optical assembly 115).

In some embodiments, system 10 comprises a laser scan frequency of no less than 200 kHz, a pullback speed of no less than 60 mm/sec or 100 mm/sec, and/or a rotation rate of no less than 250 Hz. System 10 can be configured to allow imaging of at least 50 mm of a vessel, such as at least 50 mm imaged in no more than 0.5 seconds, with no less than 800 scan lines per rotation, with approximately 400 μm pitch and/or a frame density of at least 2.5 frames/mm, and/or at least 5.0 frames/mm. In some embodiments, system 10 is configured to perform a pullback during a resting portion of the heart cycle to minimize motion artifacts. In some embodiments, system 10 comprises a rotation rate of up to 400 kHz, such as no less than 250 kHz, 300 kHz, or 350 kHz.

In some embodiments, system 10 is configured to perform a pre-treatment imaging procedure (e.g. of a tight lesion or otherwise) and provide OCT-guided treatment due to the following characteristics of system 10: processor 52 is configured to identify (e.g. via algorithm 51) a reflection generated at the splice interface between optical assembly 115 and optical core 110. The optical interface between optical assembly 115 (e.g. optical assembly 115 comprising a GRIN lens) and optical core 110 (e.g. optical core 110 comprising a NZDS fiber) can comprise a relatively large index mismatch, providing a clearly differentiable reflection. This reflection can provide a reference point for the OCT image data collected by system 10. In some embodiments, the interface can be identified by algorithm 51 with or without rotating optical core 110.

Referring now to FIG. 4, a flowchart of a method of planning a treatment procedure for a patient is illustrated, consistent with the present inventive concepts. Method 1000 of FIG. 4 is described using system 10 described in reference to FIGS. 1-3.

In Step 1100, a patient is selected for potential treatment.

In Step 1200, an OCT probe, such as imaging probe 100, is inserted into the patient.

In Step 1300 October data is gathered, such as is described herein. In some embodiments, non-OCT data is also gathered, such as patient physiologic data recorded by one or more physiologic sensors of system 10, and/or angiography data gathered by system 10.

In Step 1400, treatment information is produced, such as by system 10 and/or by a user of system 10 (e.g. the clinician of the patient performing the data gathering procedure of method 1000 or performing the subsequent treatment on the patient based on the treatment information produced via method 1000). In some embodiments, treatment information provided by system 10 is based on a combination of OCT data and non-OCT data.

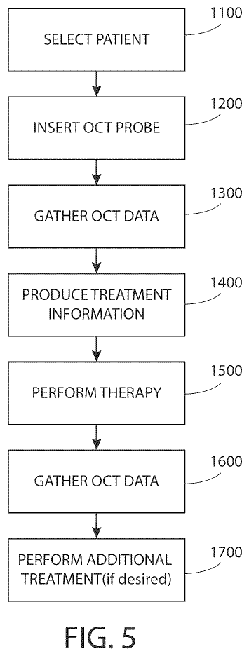

Referring now to FIG. 5, a flowchart of a method of planning a treatment procedure, performing a treatment, and assessing the treatment, is illustrated, consistent with the present inventive concepts. Method 1000 of FIG. 5 is described using system 10 described in reference to FIGS. 1-3.

In Step 1100, a patient is selected for potential treatment.

In Step 1200, an OCT probe, such as imaging probe 100, is inserted into the patient.

In Step 1300 October data is gathered, such as is described herein. In some embodiments, non-OCT data (e.g. at least angiographic data) is also gathered in Step 1300, such as is described herein.

In Step 1400, treatment information is produced, such as by system 10 and/or by a user of system 10 (e.g. the clinician of the patient performing the data gathering procedure of method 1000 or performing the subsequent treatment on the patient based on the treatment information produced via method 1000).

In Step 1500, a treatment procedure is performed on the patient, such as is described herein.

In Step 1600 October data is gathered, such as is described herein. In some embodiments, non-OCT data (e.g. at least angiographic data) is also gathered in Step 1600, such as is described herein.

In Step 1700, additional treatment can be performed, such as an additional treatment performed based on the OCT data gathered in Step 1600.

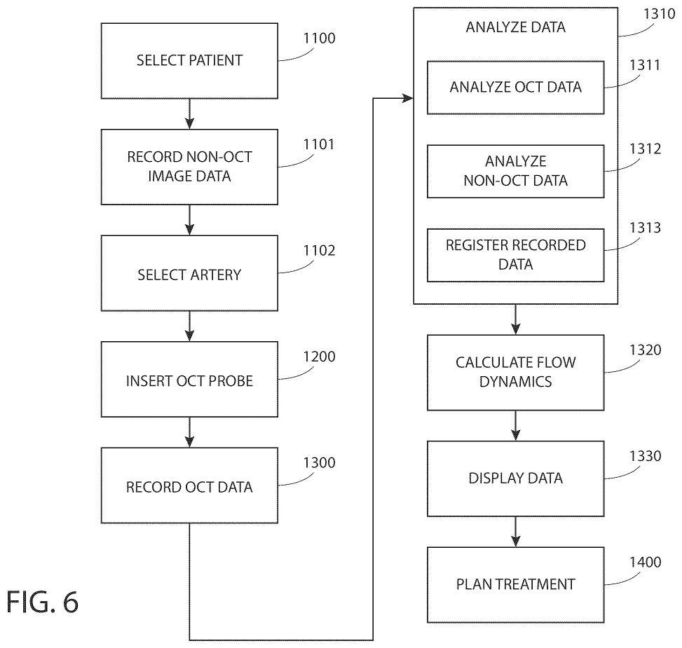

Referring now to FIG. 6, a flowchart of another method of planning a treatment procedure for a patient is illustrated, consistent with the present inventive concepts. Method 1000 of FIG. 6 is described using system 10 described in reference to FIGS. 1-3. The method of FIG. 6 is described where system 10 is used to create image data from one or more arteries of the patient's heart. It should be considered within the spirit and scope of this application that a similar method can be applied to the other blood vessels and/or other locations of the patient's anatomy (e.g. arteries and/or veins of the patient's brain or peripheral vasculature).

In Step 1100 a patient is selected. In some embodiments, the user can enter one or more patient parameters to system 10, such as via user interface 55 of console 50. The one or more patient parameters can be selected from the group consisting of: patient weight; existence of one or more patient diseases (e.g. in addition to cardiovascular disease such as diabetes if applicable); gender; age; height; TIMI score; previous coronary interventions such as stent implants, bypass grafts, and the like; and combinations of one or more of these.

In Step 1101, system 10 can be used to capture non-OCT imaging data of the selected patient, such as via second imaging device 15 described herein. As used herein, “non-OCT data” shall include but not be limited to: angiography image data; ultrasound image data; MRI image data; PET Scan image data; and/or other non-OCT imaging data. In some embodiments, non-OCT data comprises two or more of: angiography image data; ultrasound image data; MRI image data; PET Scan image data; and/or other non-OCT imaging data. In some embodiments, non-OCT data comprises angiography image data, and one or more of: ultrasound image data; MRI image data; PET Scan image data; and/or other non-OCT imaging data.

This non-OCT data can be stored in memory of system 10, such as memory of processor 52 of console 50. For example, angiography images of one or more vessels can be obtained using contrast injections, wherein the angiography data is saved in system 10 memory. Alternatively or additionally, the user may manually enter relevant patient data (e.g. data similar to and/or extracted from angiography data or other non-OCT data) into console 50 (e.g. via a keyboard or other user input component 57 of user interface 55). Step 1101 can be performed (e.g. repeated, in whole or in part) in any of the steps of the method of FIG. 6 described herein.

In Step 1102, one or more arteries, veins, and/or other conduits (“artery” or “arteries” herein) are selected for diagnosis by the user (e.g. a clinician of the patient), such as an artery which may require interventional treatment. In some embodiments, the selected vessel (e.g. a selected artery or selected vein) is manually input into system 10, such as by a user of system 10. In some embodiments, the selected vessel comprises an artery selected from the group consisting of: left circumflex (LCx); right coronary artery (RCA); left anterior descending (LAD); and combinations of one or more of these. In some embodiments, data is input into system 10 by a user during a processing step, such as Step 1310 described herein. For example, while system 10 (e.g. algorithm 51) performs one or more background calculations, system 10 can be configured to accept user input such that overall procedure time is reduced (e.g. while system 10 is processing data, the user performs required and/or other data entry).

In Step 1200, imaging probe 100 is inserted into the selected vessel (referred to herein as the “selected vessel”, the “imaged vessel”, the “selected artery”, the “imaged artery”, the “selected vein”, or the “imaged vein”, as appropriate). When two or more arteries are selected for diagnosis, the following steps can be repeated for each selected artery. Probe 100 placement (e.g. positioning of optical assembly 115) is performed to effectively obtain the images required for one or more various calculations to be performed by system 10 (e.g. flow calculations). The anatomical location at which pullback is to start (e.g. the position of optical assembly 115 at the start of a pullback) is selected to be a location beyond (i.e. distal to) the distal end of the distal-most diseased portion of the artery (e.g. the distal-most portion of a lesion). The location at which the pullback is to end (e.g. the position of optical assembly 115 at the end of a pullback) is selected to be a location within the most distally placed delivery catheter 80 through which imaging probe 100 is inserted (e.g. a distal portion of a delivery catheter 80).

In Step 1300, a pullback is performed in which imaging data comprising OCT data is recorded while probe 100 is retracted through the lumen of the selected artery.

In Step 1310, the OCT recorded data is analyzed. In some embodiments, both OCT data and non-OCT data (e.g. angiography data) is analyzed (e.g. OCT data is analyzed in combination with non-OCT data). Steps 1311, 1312, and/or 1313 (or portions of these) can be performed sequentially, simultaneously, and/or they can be interleaved as the recorded data is analyzed by system 10.

In Step 1311, recorded OCT data is analyzed by system 10. In some embodiments, the OCT data can be analyzed to identify one or more of the following: lumen boundaries; side branches; healthy (e.g. non-diseased) portions of lumen; diseased portions of lumen; type of disease imaged; the location of a guidewire within the image; and combinations of one or more of these. In some embodiments, the guidewire is removed from the OCT data. In some embodiments, system 10 identifies healthy sections of the imaged artery based on the OCT data. Healthy sections can be determined by identifying visible intima, media, and/or adventitia layers within the OCT data. In some embodiments, the myocardial mass can be estimated based on the diameter of one or more identified healthy sections of the imaged artery. In some embodiments, the estimate of the myocardial mass can be based on both OCT data and non-OCT data (e.g. angiography data).

System 10 can comprise a weighting function (e.g. where algorithm 51 comprises the weighting function), where the weighting function is configured to prioritize data (e.g. prioritize a data type) in one or more calculations, for example to preferentially bias a calculation based on OCT data vs non-OCT data, or vice versa. In some embodiments, system 10 (e.g. via algorithm 51) identifies the presence of disease proximate one or more side branches of an imaged artery. For example, if disease is detected proximate a side branch (e.g. disease is detected inside the side branch), the weighting function can be configured to prioritize angiography data related to the diseased side branch (e.g. to preferentially bias a calculation toward weighting angiography above OCT data).

System 10 can be configured to calculate (e.g. via algorithm 51) the branch angle of a side branch from the imaged artery. In some embodiments, the branch angle is used by algorithm 51 to calculate the side branch vessel diameter. System 10 can be configured to reconstruct at least a portion of the side branch from the OCT data (e.g. from the image slices of the OCT data), and/or from non-OCT data (e.g. from angiography data). In some embodiments, system 10 is configured to calculate the relationship between the side branch angle and the diameter of the size of the side branch (e.g. the size of the side branch relative to the size of the imaged artery). In these embodiments, if the relationship between branch angle and branch diameter is outside of an expected range, system 10 can be configured to “flag” this anomaly (e.g. identify the anomaly and store the associated information), and/or to alert the user of this anomaly, such as in Step 1330 described herein.

In some embodiments, system 10 is configured to identify a portion of OCT data representing the minimal lumen diameter (e.g. a portion of healthy tissue with the minimal lumen diameter). Additionally, system 10 can be configured to identify between two and five sections of the OCT data along the length of the lumen imaged during the pullback (e.g. equally spaced sections along the length of the lumen imaged). The identified sections can be used to estimate the non-diseased vessel size (e.g. lumen diameter) of the selected artery. In some embodiments, at least one of the identified sections comprises a proximal section, and at least one of the sections comprises a distal section (e.g. proximal and distal sections proximate the proximal and distal ends, respectively, of the lumen imaged). In some embodiments, the proximal and/or distal sections are within the proximal and distal 10%, respectively, of the length of the lumen imaged. In some embodiments, a weighting function is configured to apply a weight (i.e. a weighting factor) to each section based on a confidence level of the OCT data for that section. Confidence can be determined in several ways, for example the proportion of unambiguous lumen detected in a single frame (for example, at least 75% of the circumference should be unambiguous for high confidence), and/or the amount of change in lumen area from frame to frame (e.g. discontinuous frames should be given less weight). Confidence is also obtained from the deviation of circularity of the cross section of the imaged vessel.

In some embodiments, system 10 (e.g. via algorithm 51) identifies areas of disease based on OCT data and/or non-OCT data. In some embodiments, types of disease can be identified by system 10, such as when system 10 identifies plaque composition of a diseased segment of an artery.

In Step 1312, non-OCT data (e.g. angiography data) can be analyzed. In some embodiments, non-OCT data is analyzed to identify one or more of the following: vessel geometries (e.g. curves, tapers, and/or trajectories); side branch locations (e.g. the size and position of side branches of one or more main arteries to be diagnosed); vessel lengths; vessel diameters; and combinations of one or more of these. In some embodiments, the non-OCT data can be analyzed to estimate one or more of the following: myocardial mass; collateral flow; size of the heart area (myocardium) being supplied by an artery (e.g. a selected artery); and combinations of one or more of these. In some embodiments, the non-OCT data analyzed in Step 1312 comprises angiography image data and one or more of: ultrasound image data; MRI image data; PET Scan image data; and/or other non-OCT imaging data. In some embodiments, non-OCT data comprises angiography data which is converted by system 10 into QCA data. In some embodiments, non-OCT data comprises PET-scan data that system 10 converts into myocardial damage data (e.g. where system 10 adjusts vessel size in a damaged area).

In Step 1313 October data and Non-OCT data (e.g. angiography data) can be registered (e.g. correlated). In some embodiments, the data can be registered using the location, size, and shape of one or more side branches of the selected artery. In some embodiments, system 10 can comprise a digital model of the expected branching of one or more of the major vessels of the heart, for example the LCx, RCA, and/or LAD arteries. In some embodiments, the digital model is used by system 10 to register the data.

In Step 1320, cardiovascular flow dynamics can be calculated based on the analyzed data (e.g. the OCT data and/or Non-OCT data collected and/or analyzed). In some embodiments, system 10 is configured to estimate microvascular resistance distal to the selected artery.

In some embodiments, the measured size of the imaged artery is adjusted by system 10 (e.g. the size of the image artery as determined in step 1310 is adjusted by algorithm 51). System 10 can adjust the measured size of the imaged artery based on Murray's Law. For example, system 10 can assume constant sheer stress in all non-diseased areas of the imaged artery. This adjustment can minimize the error function of flow calculated by system 10 using Murray's Law. In some embodiments, flow calculations are adjusted using a weighting function that is based on the confidence of the analyzed data. For example, if Murray's law suggests an adjustment to the measured size and the confidence of the imaged cross section is relatively low then system 10 can be configured to adjust the flow calculations based on the Murray's law suggested adjustments. Alternatively, if the confidence is high, one or more adjustments based on Murray's law can be ignored by system 10.

The size of the myocardium being supplied can be estimated by system 10 (e.g. using algorithm 51) based on the selected artery vessel type and/or calculated size. In some embodiments, the estimated myocardial size can be compared to the estimated size calculated based on non-OCT Data (e.g. angiography data), such as described in Step 1312. In some embodiments, if the Non-OCT-based estimate and the OCT-based estimate vary, the user is alerted by system 10 (e.g. via a user display 56 or other user output component of interface 55). In some embodiments, system 10 accepts user input to adjust the estimate (e.g. via user input 57 of user interface 55). In some embodiments, the OCT-based estimate is given a higher weight (e.g. by a weighting function of system 10) than the angiography-based estimate (e.g. the OCT-based estimate is favored by system 10). In some embodiments, the data displayed in step 1330 can comprise a color map (e.g. a color map shown in display 56). In some embodiments, the color map can indicate the estimated myocardial size being supplied by a selected artery (e.g. the amount of myocardial tissue supplied by a selected artery). In some embodiments, hyperemic microvascular resistance is estimated by system 10 (e.g. via algorithm 51) for all or at least a portion of the heart outside the imaging area.

In some embodiments, the pressure throughout the vasculature of the heart (the “coronary tree”) is calculated. In some embodiments, fractional flow reserve (FFR) is calculated (e.g. calculated from the lowest pressure). The FFR can be calculated for a selected artery, such as for each selected artery (e.g. each artery selected and imaged using OCT). In some embodiments, system 10 can be configured to alert the user (via user interface 55) if one or more of the following is detected: significant collateral flow is found (e.g. flow above a system 10 threshold); a myocardial estimate varies significantly (e.g. the variance is above a system 10 threshold) between an OCT-based estimate and a non-OCT-based estimate (e.g. angiography-based estimate); a TIMI score indicates compromised myocardial tissue; a region of no-flow is detected angiographically (e.g. indicating a blockage); and combinations of one or more of these.

In Step 1330 October data and/or non-OCT data (e.g. angiography data) is displayed (e.g. on display 56). In some embodiments, the data is shown in an overlay arrangement (e.g. OCT data is overlaid on angiography data and/or other non-OCT data), such as after the multiple sets of data have been registered in Step 1320. In some embodiments, a graph of lumen diameter is displayed. The lumen diameter can comprise the hydraulic diameter (e.g. four times the area of the lumen divided by the perimeter). In some embodiments, one or more side branches (or portions thereof) of the selected artery are displayed. In some embodiments, the user can add data (e.g. manually add data) to system 10 indicating that disease is present proximate the displayed OCT data, for example at the proximal or distal end of the lumen, or within a side branch (e.g. the user visually determines that disease is present via information provided by second imaging device 15 and manually enters associated disease type and/or disease location data into system 10). In some embodiments, calculated vessel sizes are displayed along with the OCT images and/or non-OCT images (e.g. angiography images). In some embodiments, system 10 allows the user to edit the displayed calculated values (e.g. in order to allow a user-initiated manual adjustment of a system 10 calculated value).

In Step 1400, a user of system 10 can plan a treatment procedure based on the displayed data. In some embodiments, the user indicates a length and location of a “treatment area” to be evaluated by system 10 (e.g. the user indicates the treatment area by clicking on the displayed image). In some embodiments, system 10 displays the estimated vessel diameter at the proximal and distal ends of the selected treatment area. In some embodiments, the vessel diameters are estimated by system 10 (e.g. via algorithm 51) using Green's Theorem. In some embodiments, system 10 displays one or more pieces of information related to the selected treatment area, for example any warnings associated with the treatment area and/or the plaque composition within the treatment area. In some embodiments, post-treatment flow dynamics (e.g. FFR) can be estimated assuming the planned treatment (e.g. stenting) opens the treated vessel to the estimated diameter at the proximal and distal ends of the selected treatment area (e.g. where two diameters can be used to indicate vessel tapering). In some embodiments, the post-treatment flow dynamics can be estimated based on the calculated pre-treatment flow dynamics and the proposed treatment. In some embodiments, the user can vary the proposed treatment area, and system 10 can update the flow dynamics based on the new treatment area. In some embodiments, the user can indicate two or more treatment areas (e.g. two or more non-contiguous treatment areas), and post-treatment flow dynamics can be estimated for each treatment area.

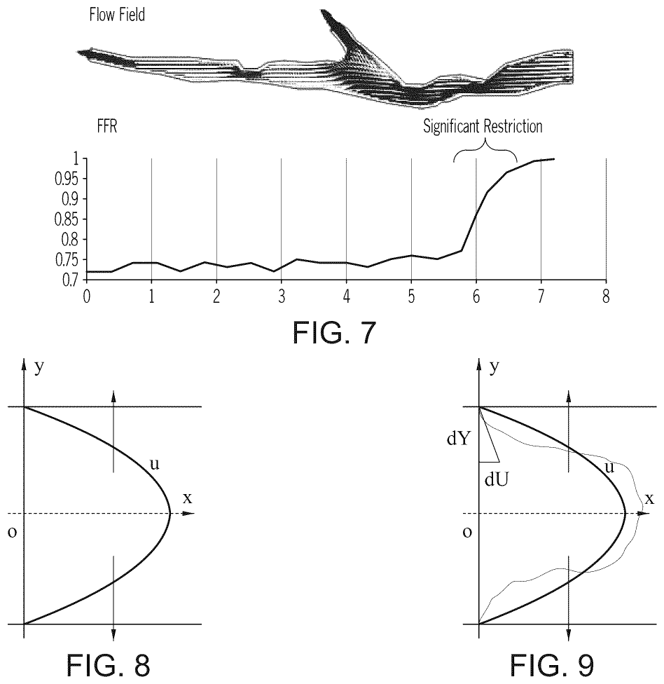

As described herein, system 10 can be configured to calculate the flow characteristics through one or more blood vessels that have been imaged using probe 100 (e.g. via one or more pullbacks). System 10 can be configured to calculate the flow dynamics (e.g. flow field and/or pressure drop) through an imaged vessel (e.g. an artery to which a treatment has been or will be performed). In some embodiments, system 10 calculates the flow and pressure drop of coronary vessels using a full 3D Navier-Stokes simulation of the vessel imaged using probe 100. As shown in FIG. 7 and described herein, system 10 can be configured to include in its analysis (e.g. via algorithm 51) numerous geometric and other features of the imaged area, such as when every significant morphological feature is represented. System 10 can directly measure the pressure in the vessel, and the FFR at the locations along the length of the vessel may be calculated, as described herein. Regions where the rapid changes in FFR occur (e.g. as identified by system 10) can be used to justify intervention (e.g. stenting).

FIG. 7 illustrates a full Navier-Stokes simulation of flow through an artery, and the FFR calculated through the length of the artery.

The use of a full 3-D Navier-Stokes equation is computationally intensive and can require a time duration that is undesirably long. In some embodiments, system 10 utilizes a two dimensional (2-D) scheme that gives results that are sufficiently accurate for the calculation of the FFR, yet require less time than a full three dimensional (3-D) Navier-Stokes-based approach.

In some embodiments, system 10 can be configured to use 3-D Navier-Stokes equations in subsets of a flow field, such as when small morphological features effect the flow. Examples of these include: bifurcation lesions with significant stenosis; locations downstream of a constriction, such as to predict the transition to turbulent flow. These short lengths of vessel can be computed relatively quickly, and the calculated flow resistance can be modeled as resistor elements in the 2-D scheme.

System 10 can be configured to utilize a 2-D scheme that uses the lumen area calculated from OCT data vs the distance and a flow shape parameter to predict pressure drop, flow separation and viscous wall forces.

For fully developed flow in pipes, the relationship between flow and pressure drop is:

$$Q = \frac{- {πR}^{4}}{8 µ} \frac{dP}{dx}$$

$$\text{where:}$$

$$Q = Volumetric flow rate$$

$$R = Radius of the pipe ( one half the pipe diameter )$$

$$µ = blood viscosity$$

$$dP / dx = \text{Pressure drop} in \text{the pipe the pipe direction}$$

In some embodiments, in order to increase the speed of the calculations performed by system 10, μ is set to a constant (e.g. 3 centipoise), in other words the shear thinning effects of blood are ignored. For correlations to measured FFR, system 10 can be configured to adjust μ to hematocrit, but in general use it is taken as constant.

The velocity profile thru the pipe is shown in FIG. 8. The profile is parabolic, with the highest velocity at the center.

When the diameter of the vessel changes, the velocity profile will change from parabolic to some other shape. In some embodiments, system 10 utilizes a one dimensional (1-D) method, using just these two continuous parameters along the length of the vessel to calculate pressure drop, the vessel diameter and the deviation from the parabolic shape. System 10 calculates other continuous values along the length based on these two parameters. These values can include Reynolds Number, degree of turbulence, rate of pressure gradient change and wall shear stress.

In regions where the vessel diameter is rapidly getting larger, the velocity near the walls decreases as shown in FIG. 9. The shear stress μ dU/dy (evaluated at the wall), decreases, which decreases the pressure drop component from viscosity.

In some embodiments, system 10 performs a 1-D solution comprising a fourth order, axially symmetric equation to represent the velocity:

$$U ( y ) = a + by + {cy}^{2} + {dy}^{3} + {ey}^{4}$$

The coefficients a, b, c, d, and e are calculated along the length from the upstream velocity profile and the rate of pressure change. The coefficients of the higher order terms (third and fourth) may be kept in bounds to keep the solution stable. This limitation greatly speeds up the calculations with minimal loss of precision.

In some embodiments, system 10 uses a 1-D method to calculate the wall shear from the shape function.

In some embodiments, system 10 uses a 2-D method to calculate shear stress directly from the velocity gradient at the wall. For example, the 2-D method can use the polar coordinate form of the Navier-Stokes equation to simplify the calculations. All low Reynolds Number terms can be used. Viscous dissipation terms can be avoided (e.g. not used). The equations are taken at steady state, thus the time dependent terms are not used. Also, gravity can be ignored as it is not significant in this single phase flow.

$$\begin{matrix}( V \cdot ∇ ) V & = & - ∇ p & + & µ {∇}^{2} \vec{V} \\ ↖ & & ↗ & & ↖ \\ Convective & & Pressure gradient & & Diffusion term\end{matrix}$$

The pressure gradient is solved in the radial and axial directions as is the velocity equation. The continuity equation is also solved, allowing for radial flow. The radial flow velocity is insignificant and may be ignored for wall shear:

$$∇ \cdot \vec{V} = 0$$

System 10 can include any number of efficient “flow solvers”. In some embodiments, system 10 includes a finite volume solver. A 2-D discretization can be performed. Smaller cells can be used in areas of rapidly changing geometry. Flow can be stepped downstream starting from parabolic flow at the inlet. The equations can be solved with an assumed pressure gradient and once the entire flow field is calculated with that pressure, system 10 then recalculates the pressure with the updated flow field. System 10 can be configured to iterate the solution until the force balance in each finite volume is converged to pre-defined tolerance.

In some embodiments, one or more steps are performed to speed up calculations. For example, the length and width of the cells can be increased in areas where the vessel does not change shape. Alternatively or additionally, a pressure drop can be automatically found from the calculated pressure field, such as when the pressure drop includes both the losses from viscosity and momentum. In some embodiments, to speed calculation times, system 10 can calculate the initial pressure field from 1-D equations based on area changes along the length. The equations of Kirkeeide (see Kirkeeide, R. L. (1991). Coronary obstructions, morphology and physiological significance. In Quantitative Coronary Arteriography (pp. 229-244). Kluwer Academic1) are especially suited for this application. Areas of reduced cross section are treated as the restrictions in his model. An individual “restriction” is considered a reduced cross section separated by number of diameters away from the next restriction, generally 5 to 10 diameters. If the restrictions are closer than this, then an estimate can be made of their interaction. The equations of Banerjee (see Banerjee, M. K., Nag, D., Ganguly, R., & Datta, A. (2008). Stenotic interaction on hemodynamic parameters in double stenoses. International Journal of Computational Fluid Dynamics, 22 (9), 609-622. doi: 10.1080/10618560802372033) can be used to estimate the interaction.

System 10 can be configured to calculate transition to turbulence from system 10 based correlations related to vessel wall divergence angle, adverse pressure gradient, Reynolds Number and the deviation of the flow profile from the parabolic shape. For all the calculations, the variation through the heart beat can be ignored (e.g. for coronary blood vessel analysis). In these embodiments, the principal concern is the wave free time during diastole when turbulent flow is the most likely.

Once transition to turbulence is determined by system 10, a separate turbulent model can be used. The 2-D Navier-Stokes equations can be (still) solved, but an artificially higher viscosity can be used. The K-Epsilon model can be used to calculate the length of the turbulent section. When the dissipation term drops below a preset threshold of system 10, the entire cross section is returned to laminar flow. The 1-D model can also include tests for transition to turbulence with the associated pressure drop increase.

For the 2-D model, if separation at the walls causes an unsteady solution, the reverse flow is not calculated, and the Navier-Stokes Equations are no longer used. Instead, the pressure drop for fully developed turbulent flow is used. The default length of this section is taken as five diameters and is shortened or lengthened based on whether the geometry is converging or diverging, and the Reynolds number of the flow. After that region, system 10 assumes a laminar parabolic velocity to enter the next section.

A transition to turbulence or a separation of flow along the wall generally means that an intervention needs to be performed (e.g. stenting). Thus, for the calculation of the decision to stent, correct calculation of the transition to turbulence is more important than the accurate calculation of the pressure drop in the turbulent section.

System 10 can be configured to convert non-circular sections to circular cross sections, for the purpose of these calculations. The hydraulic diameter, which is four times the cross-section's perimeter divided by the area, is used for the circular cross section diameter.

For the purposes of calculating FFR, system 10 can set the arterial pressure to 90 mm Hg and the venous pressure to 30 mm Hg. This pressure difference is the driving force for the flow. The FFR at any location is calculated as the local static pressure divided by the arterial pressure.

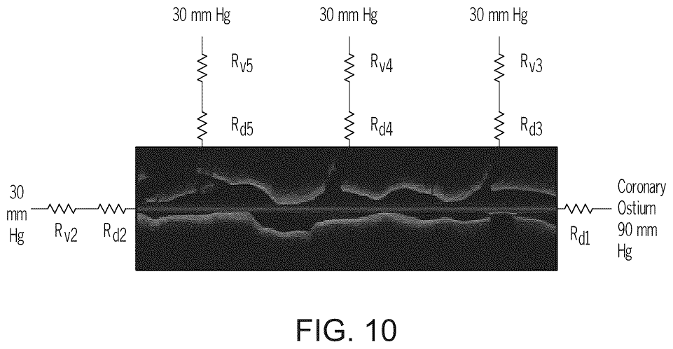

Referring now to FIG. 10, a vessel flow schematic is illustrated. If there is disease at a side branch, the unimaged branch resistance from disease, Ra₂, is calculated by system 10. This resistance can be based on the calculated disease shape. The resistance can be estimated from the algebraic equations of Kirkeedee. The microvascular bed resistance Ry for each side branch in the OCT image is put in series with Ra for the diseased branches. Side branches below 1 mm diameter can be ignored as they don't significantly contribute to the flow. If the vessel ostium (e.g. the ostium of the imaged vessel, or an ostium proximal to the imaged vessel, vessel ostium herein) isn't imaged, a resistance is assigned to it, Rdi, based on other imaging (e.g. angiography) of the vessel ostium, if available, or based on a standard ostium shape, if not. The distal end of the image is assigned a microvascular resistance of Rv₂, and diseased resistance assigned to it, Ra₂, (if there is disease distal to the imaged area). Raz can be estimated from angiography or from a more distal OCT pullback.

System 10 can assume (e.g. for calculations) an initial flow at the vessel ostium of 3 ml/s for an artery on the right side of the heart, and an initial flow at the vessel ostium of 4 ml/s for a vessel on the left side of the heart. Starting from the vessel ostium, system 10 can determine an initial flow estimate by splitting up the ostial flow based on branch resistances, with no resistance applied to the imaged area. The imaged area can be broken into sections divided at each side branch as well as the distal and proximal ends. Then, Kirkeeide's and Banerjee's equations can be applied to each section and resistance at that flow can be calculated. Flow in each section can then be calculated from an explicit equation. The resistances can then be adjusted for that flow, and the flow recalculated from Kirkeedee's equations until a converged solution is obtained. If the calculated flow goes above 6 ml/s, the flow can be limited to a 6 ml/s maximum as these higher flows are not physiologic (i.e. not expected). A high flow estimate can be due to a slight misestimate of the microvascular resistance. This slight error does not have a significant effect on the FFR results as they relate to FFR values upon which treatment (e.g. stenting) decisions are made.

The flow and pressure at each side branch are the output of this calculation performed by system 10. The calculated pressure is used by the Navier-Stokes 2-D solver with a linear interpolation of pressure between each side branch. The non-imaged section(s) can still use Kirkeedee's and Banerjee's equations for Rd. System 10 can use the 1-D solver as an option.

To create an image using probe 100, blood must be flushed from the vessel being imaged. Currently available OCT catheters have a diameter that blocks a large part of the vessel, making decisions about whether to intervene (e.g. stent) difficult. Probe 100 of the present inventive concepts can comprise a significantly reduced diameter (e.g. a distal portion of no more than 0.030″, no more than 0.023″, no more than 0.020″, and/or no more than 0.016″). Combined with a 0.014″ guidewire, probe 100 and the guidewire take up no more than 0.044″ of the vessel.

An FFR of 0.8 is generally accepted as the value above which the vessel is not to be treated (e.g. stented). To make decisions whether or not to stent an artery, OCT must be safe and effective at imaging vessels above and below this cutoff FFR. The cutoff FFR of 0.8 is associated with an MLA of 3.07 mm² (see Waksman, R., Legutko, J., Singh, J., Orlando, Q., Marso, S., Schloss, T., . . . . Torguson, R. (2013). FIRST: Fractional Flow Reserve and Intravascular Ultrasound Relationship Study. Journal of the American College of Cardiology, 61 (9), 917-923. doi: 10.1016/j.jacc.2012.12.012). For a round vessel, this correlates to a 1.98 mm diameter vessel. Currently, the smallest commercially available OCT catheter is 2.6 F. Using this device and a 0.014 guidewire, most of the vessel (1.23 mm) is taken up by the combined devices. Occluding the vessel with this large of a catheter and guidewire makes it difficult to flush blood from the vessel to get a clear image, and otherwise complicates the imaging procedure.

System 10 can incorporate (e.g. take into account) the disease of the artery being investigated (e.g. at least imaged). If there is disease distal or proximal to an imaged area, its resistance to flow would need to be estimated, such as from angiography data, which is not as accurate as OCT data provided by system 10. Probe 100 and retraction assembly 800 can be configured to provide up to a 10 cm pullback, which can image from the vessel ostium to locations beyond the diseased areas for most coronary lesions (e.g. all lesions on the left side and for most of the lesions on the right side). System 10 can be configured to perform the pullback at a rapid rate, such as a rate that is faster than commercially available systems. System 10 (e.g. retraction assembly 800) can be configured to perform a pullback at a rate of 50 mm/sec (i.e. 10 cm in two seconds), such as with a 0.2 mm frame spacing. Two seconds is about the maximum time a vessel can be cleared with a safe dose of contrast (e.g. correlating to about 14 ml). Thus, probe 100, for almost all coronary lesion locations, is able to capture continuous images between a location distal to the lesion(s) and the vessel ostium. The 0.2 mm frame spacing provides sufficient resolution in imaging a stenosis to accurately calculate FFR.

System 10 can be configured to provide a long scan range, such as a scan range of up to 8.7 mm, such as to provide better identification of side branches. Typical coronary arteries do not exceed 5 mm in diameter (e.g. the left main is typically 5 mm diameter). System 10 can identify (e.g. via algorithm 51) a side branch, such as by the detected wall being significantly further away than the surrounding wall. Without a long scan range (e.g. a scan range that only reaches to the wall), side branch detection is more difficult.

The long scan range provided by system 10 also improves on estimating the diameter of a side branch. Imaging a significant length of side branch (at least 2 mm) allows finding the side branch direction and thus providing a better estimate of its diameter. The side branch size is correlated with branch angle. The larger the side branch, the smaller the angle to the main vessel. As disease tends to occur at bifurcations, system 10's ability to image beyond the bifurcation to determine the non-diseased diameter of the side branch, improves the estimation of the size of the vascular bed supplied by the side branch and thus the microvascular resistance assigned to that side branch.

The diameter of imaging probe 100 is sufficiently small to allow imaging of stenotic vessels or otherwise reduced diameter vessels in a pre-treatment procedure. Probe 100 can comprise a diameter of less than or equal to 1.7 F (0.022″). An imaging probe comprising a diameter of less than or equal to 2.2 Fr (0.029″) allows for pre-intervention OCT imaging (e.g. can be used to image a reduced diameter portion of a blood vessel). In some embodiments, system 10 comprises a frame rate of 250 Hz, which allows for rapid pullbacks of extended lengths that can be used to capture lesion information, as well as information about the vessel ostium, in a single pullback. In some embodiments, system 10 also provides a scan range of no less than 6 mm, which allows for side branch detection and size estimation.

Fibrotic tissue, calcium, and lipids each have distinct attenuation and brightness values (e.g. when imaged by imaging probe 100 of system 10). Calcium is imaged via OCT as an area with a low backscattering coefficient and low attenuation; lipid shows high-attenuation and high-backscattering; and fibrotic tissue shows low attenuation and high backscattering (see Xu, C., Schmitt, J. M., Carlier, S. G., & Virmani, R. (2008). Characterization of atherosclerosis plaques by measuring both backscattering and attenuation coefficients in optical coherence tomography. Journal of Biomedical Optics, 13 (3), 034003. doi: 10.1117/1.2927464; see also Yabushita, H., Bouma, B. E., Houser, S. L., Aretz, H. T., Jang, I., Schlendorf, K. H., . . . . Tearney, G. J. (2002). Characterization of Human Atherosclerosis by Optical Coherence Tomography. Circulation, 106 (13), 1640-1645. doi: 10.1161/01.cir.0000029927.92825.f6). Normal, non-diseased tissue can be identified by its three characteristic layers of the media, adventitia, and intima (see Tearney, G. J., Regar, E., Akasaka, T., Adriaenssens, T., Barlis, P., Bezerra, H. G., . . . . Weisz, G. (2012). Consensus Standards for Acquisition, Measurement, and Reporting of Intravascular Optical Coherence Tomography Studies. Journal of the American College of Cardiology, 59 (12), 1058-1072. doi: 10.1016/j.jacc.2011.09.079).

In some embodiments calcium and/or lipid deposits are automatically identified by system 10, such as by using 1-D (e.g. A-line classification), 2-D, and/or 3-D methods to analyze the OCT data. Machine learning approaches such as deep learning (e.g. convolutional networks) and/or supervised classification (e.g. support vector machine or random forest) can be used (e.g. by algorithm 51 of system 10) to automatically classify tissue types in OCT images. Image features such as optical backscattering, optical attenuation, layer analysis and texture analysis can be used. In some embodiments, automated and/or manual detection of calcium, lipid and normal tissue layers allows for improved calculation of flow (e.g. FFR) and/or procedure planning (e.g. determining the proper position of a stent to be implanted).

An estimate of microvascular resistance can be made by system 10, such as an estimation based on the size of a non-diseased vessel. Identification of the non-diseased sections would help microvascular resistance calculations. In some embodiments, the user can manually identify and/or correct the identified non-diseased sections, such as via user interface 55. In some embodiments, if too few non-diseased sections are identified in an OCT image (e.g. none or a limited number of normal sections can be found), algorithm 51 of system 10 can use information from one or more diseased sections for estimating microvascular resistance.

When treating cardiovascular disease, stents are sometimes placed across all diseased areas. Ideally the stent begins and ends in normal tissue. A stent placement algorithm (e.g. algorithm 51 of system 10) could consider not only FFR, but also one or more properties of the vessel wall. In some embodiments, stent placement algorithm 51 does not allow the user to place a proposed stent at a location that begins or terminates on diseased tissue (e.g. during a planning process, stents are only allowed to be placed spanning from healthy tissue to healthy tissue), and/or algorithm 51 warns the user that the selected stent ends are in a diseased portion of a vessel. Studies have shown that stenting onto lipid can be dangerous, as it can cause an embolic event. In some embodiments, if lipids are detected at one or more locations to be covered by a stent, a stent placement-based algorithm 51 prevents or alerts the user about positioning either end of the stent in lipid.

It may be difficult to fully dilate a vessel with a stent if calcium takes up the majority of the circumference of a wall. In some embodiments, if a significantly calcified section is identified by algorithm 51, the user is alerted that stenting may not be sufficient to open the stenosis. In some embodiments, after significant calcification has been identified, system 10 provides (e.g. suggests) alternate treatment methods, such as an atherectomy procedure (e.g. a rotational atherectomy procedure). In some embodiments, if circumferential calcifications are identified, this information is used when calculating post-treatment FFR values when simulating results post-stent implantation. Circumferential calcification may prevent the stent from fully expanding and calcium distribution information can provide a more detailed estimated lumen area to be achieved by stenting, providing improved guidance to the user.

In some embodiments, such as when algorithm 51 is configured to estimate the size of a side branch of an imaged vessel, the non-diseased vessel size of the imaged vessel is calculated. Sections of the imaged area that are significantly below this diameter may be checked for disease. If no disease is found in these areas, the vessel is likely in spasm, and should not be stented. In these instances, system 10 can alert the user that a spasm is likely. In some embodiments, when a spasm is identified by system 10, FFR can be calculated with and without the spasm, with both values being displayed to the user by system 10 (e.g. such that the user decides whether to stent based on the displayed information).

The improved pullback (e.g. longer length and/or increased speed) of the present inventive concepts increases the imaged vessel length (e.g. a greater length of a selected vessel can be imaged than with other imaging devices). In some instances, due to difficulty clearing blood, the vessel wall cannot be detected for the entire length of the pullback. System 10 can be configured to delineate the portion of a pullback which is clear, and the ends of the delineated length can be checked for presence of normal tissue. If the tissue at the end portions is diseased, angiography data can be analyzed to determine the extent of stenosis proximal and distal to the clear portion of the pullback. Subsequently, Ra is calculated for these areas outside of the delineated pullback. Alternatively or additionally, the user can be notified that disease may exist outside of the imaged area and be prompted to enter the minimum diameter. The use of a user entered minimum diameter may not be as accurate as a more detailed lumen (as calculated from OCT data), but it allows for some input with limited information. If a portion of a pullback is unclear (e.g. due to poor blood clearing), the user can re-image the vessel. In some embodiments, the two images are combined by system 10 to create a hybrid image.

In some embodiments, the distal end of delivery catheter 80 is easily identified in the OCT data (e.g. when imaging assembly 115 is retracted into delivery catheter 80 during a pullback procedure). System 10 can assume that the vessel ostium is proximal to the distal end of delivery catheter 80, such as when system 10 assumes that delivery catheter 80 is placed a standard distance distal to the vessel ostium, such as a distance of approximately 5 mm. If there is disease at the ostium of the imaged vessel, the user may enter the minimum diameter at the ostium. The vessel display may show the anticipated size and location of the ostium. System 10 can enable the user to input a stenosed ostium's size.

In some embodiments, system 10 can comprise a digital model of the expected branching of one or more of the major vessels of the heart, such as described in reference to Step 1313 of Method 1000 of FIG. 6. Identification of major branches of the imaged vessel takes place from the vessel ostium. For example, if the LCx is imaged, the LAD is expected within 5 to 15 mm of the vessel ostium (see Abedin, Z., & Goldberg, J. (1978). Origin and length of left main coronary artery: Its relation to height, weight, sex, age, pattern of coronary distribution, and presence or absence of coronary artery disease. Catheterization and Cardiovascular Diagnosis, 4 (3), 335-340. doi: 10.1002/ccd. 1810040318). Alternatively, if the LAD is imaged, the LCx is expected in the same region. If the branch detection-based algorithm 51 is unable to detect a branch in this region, the user can be warned and/or allowed to input the branch size and location, if in fact the branch was missed.

In some embodiments, non-OCT data (e.g. angiography data) can be analyzed by system 10 (e.g. by algorithm 51 of system 10) to determine microvascular resistance. Microvascular resistance estimation can be adjusted by system 10 for the vessel selected. The right-side heart microvascular resistance is lower for the same sized vessel. Instead of or in addition to determining the microvascular resistance based on the branch diameter, the non-OCT data can be analyzed by algorithm 51 of system 10, and a vessel territory can be assigned to each branch. The right side of the heart can be more complicated because the right side may or may not supply the left side of the heart (e.g. when the patient is right-side or left-side dominated). Automated and/or manual algorithms (e.g. algorithms 51) can allow assignment of the entire heart to particular vessels. This method is most accurate when both the left and right sides are imaged, which is not always done.

In some embodiments, system 10 obtains OCT image data by placing optical assembly 115 of probe 100 within a selected blood vessel, and this data can include vessel information such as vessel geometry information, vessel location information (e.g. with respect to anatomical landmarks), and the like. Second imaging device 15 can comprise a fluoroscope, positioned outside the selected vessel, which can gather non-OCT data that is similar or dissimilar to the OCT data gathered by probe 100. The angiographic data can include data representing an entire coronary tree, but at relatively low resolution (e.g. 150 μm to 250 μm resolution), for example captured at the same time as the OCT data. The OCT data can comprise data representing much higher resolution (e.g. approximately 10 μm resolution), with frames captured at rates of 250 frames/second. The OCT data can be used to create a 3-D model of the selected vessel, over a relatively long time span (e.g. from 0.5 seconds to 4.0 seconds). Either the OCT data or the non-OCT data can be used to calculate flow. Algorithm 51 can use techniques incorporating 3-D models, as described herein, such as native 3-D (e.g. using OCT data) or constructed 3-D (e.g. cat-scans and/or bi-plane angiography data).

In some embodiments, algorithm 51 analyzes data (e.g. OCT data captured over time) and creates a 2-D image set (e.g. a series of 2-D snapshots, or image frames, at well-defined points in time). Algorithm 51 can reduce the 2-D information to a descriptive single number, such as a hydraulic diameter or similar variable. This descriptive single number can be very effective for calculating flow drops and/or pressure drops. The calculation can depend on detection of a well-defined lumen contour, and algorithm 51 can incorporate image analysis techniques, machine-learning techniques, or a combination of these. Algorithm 51 can convert time information to a distance using various techniques to reduce error. For example, high-speed pullbacks (e.g. at least 50 cm/sec), gated pullbacks, angiographic co-registration, landmark co-registration (e.g. side branch co-registration), and the like. Algorithm 51 can include side branch information in a model, such as in the form of additional resistive elements, and/or flow modifiers. Additional information can be included (e.g. OCT data and/or non-OCT data) in a 1-D or 2-D straight-line model, such as when curvatures from angiographic images are included. In some embodiments, system 10 avoids the use of externally derived vessel geometries (e.g. curvatures or other geometry information from angiography data) when calculating flow and/or pressure (e.g. based solely on OCT data), such as to reduce calculation time.

In some embodiments, system 10 captures data in the form of r, theta, and, t where r is the radial distance from the zero-point and theta is the angle relative to a defined zero-angle. It is useful to transform r and theta to rectangular coordinates x, and y; t is the timepoint of the particular snap-shot (e.g. image frame). System 10 can calculate the local derivatives (slopes) for x and y:

$${x}_{n}^{′} = {x}_{n} - {x}_{n - 1} ; {y}_{n}^{′} = {y}_{n} - {y}_{n - 1} ( or other similar method ) .$$

With x, y, x′, and y′, system 10 can calculate the area and perimeter of the vessel cross section:

$$Area = A = {\sum }_{n = 1}^{N} ( {x}_{n} {y}_{n}^{′} - {y}_{n} {x}_{n}^{′} ) ,$$

$$where N is the number of points in the circumference ,$$

$$and$$

$$Perimeter = P = {\sum }_{1}^{N} \sqrt{( {x}_{n}^{{′}^{2}} + {y}_{n}^{{′}^{2}} )} ,$$

$$\text{which is simply the sum of the} ‘ little hypotenuses ’ around the shape .$$

System 10 can calculate these results as the OCT data is collected, and from that system 10 can calculate the hydraulic diameter:
D_{H}=4A/p, which reduces to the simple diameter for a circular section. This can be used to obtain accurate flow calculations using just a 1-D number.

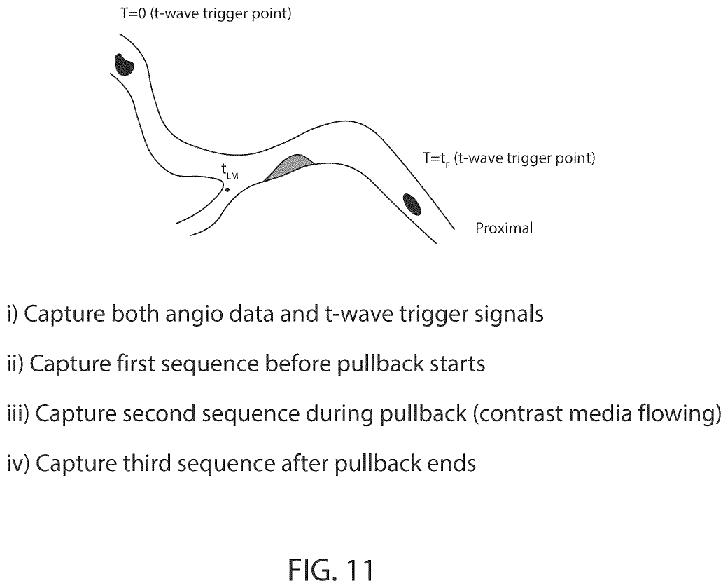

System 10 can add lengths, by turning the time-points of each snap-shot into distances. For example, for a zero-order: the pullback speed is used to convert to distance. For first order: the start of the pullback is gated (e.g. with EKG T wave) with a low motion portion of the heart cycle, and a pullback speed is used to capture the relevant section of vessel length in approximately 50% of the heart cycle or less (e.g. 30 mm to 60 mm at a 100 mm/sec pullback rate). For second order: a gated pullback can be used, with semi or fully automated simplified co-registration (single plane angiographic image), see FIG. 11. With three sequences captured (sequences ii thru iv as shown in FIG. 11), a radiopaque marker of probe 100 can be identified at the start of pullback and at the end of the pullback, and an angiography image (frame) closest in time to the respective trigger (e.g. T-wave trigger correlating to the point in the heart cycle that the tissue is relatively motionless). Note that in sequences (ii) and (iv) there is no contrast flow (e.g. the vessel walls are not visible, but the radiopaque marker is visible). With the sequence captured during pullback, sequence (iii), the vessel walls are visible, but the radiopaque marker may be obscured. This sequence provides the shape curvature information, such as by using the best projection plane standard practice, such as to minimize out-of-plane issues. The distance traversed between the radiopaque marker at sequence (ii) and (iv) is the pullback distance (e.g. a distance of 50 mm). System 10 can map this known pullback distance along the artery shape, such as in a linear fashion. If landmarks are detected in the OCT image at time tim, system 10 can add further corrections, where the distance moved is tim times the pullback speed.

In some embodiments, system 10 obtains non-OCT data comprising angiography data from second imaging device 15 (e.g. a device 15 comprising a fluoroscope). The non-OCT data can comprise sequential image frames that show the advancement of contrast agent through a blood vessel (e.g. through a coronary artery). System 10, via algorithm 51, can determine the change of contrast location from frame to frame, such as to estimate the velocity of the blood through the vessel, also referred to as the TIMI frame count, or TIMI score, where TIMI stands for thrombolysis in myocardial infarction. This frame count velocity can be for a location in the coronary tree, such as flow at the vessel ostium. System 10 can be used in an analysis using a resistor model of the coronary tree. For example, instead of a pressure estimation (e.g. 90 mmHg input pressure), the TIMI-based velocity is input into the resistor model. The input flow rate is the TIMI velocity times the vessel cross sectional area near the vessel ostium. Inlet pressure is allowed to rise or fall to cause the velocity to match the TIMI velocity. In this method, microvascular resistance does not change. System 10 can calculate FFR, as the local pressure is divided by the inlet pressure.

In some embodiments, system 10 is configured (e.g. via algorithm 51), to identify which particular vessel is the vessel being imaged (e.g. based on one or more side branches detected and/or the vessel geometric information (e.g. shape, trajectory, and/or size information)). This vessel identification can be made autonomously by system 10, and/or it can be confirmed by a user of system 10. In some embodiments, system 10 identifies the vessel, and/or confirms the vessel, using non-OCT data (e.g. using angiography data acquired from second imaging device 15 comprising a fluoroscope).

While the systems of the present inventive concepts have primarily been described in imaging and creating treatment information for arteries, and in particular coronary arteries, all blood vessels, and other body conduits should be considered within the spirit and scope of this application and its claims.

In some embodiments, system 10 is configured to compensate for cardiac motion. For example, a pullback can be performed (e.g. in a time period of no more than 0.3 seconds, or 0.2 seconds) that is synchronized to be started (e.g. via an EKG-based trigger) during a portion of a cardiac cycle that has minimal motion of heart tissue.

In some embodiments, imaging probe 100 comprises a unique identifier, such as an RFID or other identifier that can be read by console 50 (e.g. when imaging probe 100 is attached to patient interface module 200 (PIM 200)). In some embodiments, system 10 comprises a first imaging probe 100a and a second imaging probe 100b that are each identified with a unique identifier used to differentiate the two probes. In these embodiments, probe 100a and probe 100b can be of similar or dissimilar construction. For example, probe 100a and probe 100b can be of similar construction (e.g. the same construction), but each probe model results in a different configuration of system parameters (e.g. an automatic configuration that is established when the probe 100 is attached to PIM 200). In some embodiments, when probe 100a is attached to PIM 200, system 10 is enabled to calculate FFR values (e.g. on demand by a user), but when probe 100b is attached to PIM 200, FFR functionality of system 10 is disabled. In other words, an FFR-enabled probe 100 is required to enable FFR functionality of system 10, even if the only difference between an FFR-enabled probe 100a and a non-FFR-enabled probe 100b is the information included in an RFID or other model-identifying component of probe 100.

In some embodiments, when system 10 and/or probe 100 is enabled for FFR calculation, one or more system 10 parameters can be different than (e.g. modified from) a non-FFR enabled system and/or probe. For example, an FFR enabled system can be configured to rotate optical core 110 at a faster rate than a non-FFR system, such as at least 1.5 times faster, or at least 2 times faster. Faster rotation of core 110 can enable faster pullback speeds, longer pullback distances, and/or combinations of these, without a reduction in resolution of the collected image data. Additionally or alternatively, the faster rotation of core 110 enables light source 310 to deliver a higher output power without increasing tissue exposure. In some embodiments, an FFR enabled probe 100 can comprise more markers 131 than a non-FFR probe 100 includes, such as to enhance image-based tracking and/or improve registration accuracy (e.g. registration with fluoroscopy images and/or images from other imaging modalities).

In some embodiments, an FFR enabled probe 100 can be configured such that probe 100 extends beyond the tip of a delivery catheter, for example delivery catheter 480 described in reference to FIG. 1B herein. In some embodiments, probe 100 is configured to extend beyond a guidewire lumen, such as a rapid exchange guidewire tip of a delivery catheter. In these embodiments, probe 100 can be configured to capture image data distal to the distal end of a guidewire over which the delivery catheter has been advanced, such that the image data does not comprise any guidewire data (known as guidewire shadows). In some embodiments, extending probe 100 beyond the distal end of a delivery catheter allows imaging of a vessel with a smaller device (e.g. probe 100 has a smaller diameter than the delivery catheter), allowing for a minimal disruption of flow within the imaged vessel. Additionally or alternatively, the lack of any guidewire shadows in the image data allows for improved imaging, improved lumen reconstruction, and/or improved calculation of flow dynamics of the vessel.

In some embodiments, vessel identification information regarding one or more vessels to be imaged is captured by system 10, such as to associate vessel image, flow and/or other data to that identification. In some embodiments, a user enters the vessel identification information, and system 10 is configured to perform an assessment of the accuracy of this user-provided identification. For example, if system 10 determines that vessel information entered by the user is or at least may be inaccurate, an alert can be provided to the user. Alternatively or additionally, system 10 can be configured to identify a blood vessel, such as when system 10 automatically enters the blood vessel name into system 10 without user input (e.g. to save time). This automatic identification can be confirmed and/or adjusted by a user. In some embodiments, the vessel identification performed by system 10 is based on OCT data, non-OCT data (e.g. angiography information), or both.

In some embodiments, system 10 is used to image a blood vessel (e.g. create OCT data) both prior to and after a treatment procedure has been performed (e.g. a lesion has been stented). For example, system 10 can be configured to produce pre and post-treatment FFR data. In some embodiments, system 10 compares information produced based on image data (e.g. OCT data and/or non-OCT data) gathered prior to treatment, to information produced based on image data (e.g. OCT Data and/or non-OCT data) gathered after a treatment has been performed. This comparison can result in system 10 alerting the user of potential inaccuracies or other issues with the information provided by system 10. In some embodiments, probe 100 and other components of system 10 are configured to gather at least OCT data and produce a pre-treatment FFR value that is used to make a treatment decision (e.g. whether or not to stent or otherwise treat).

In some embodiments, system 10 is configured to provide information related to plaque-content of a lesion.

In some embodiments, information produced by system 10 based on OCT data and/or non-OCT data (e.g. at least angiography data) is used as “training sets” for a CT approach.

In some embodiments, information produced by system 10 is determined using OCT data and/or non-OCT data that is processed using machine learning or other artificial intelligence. In some embodiments, information produced by system 10 is determined using a cloud-based approach.

As described herein, system 10 can be configured to produce information (e.g. at least FFR values) prior to a treatment, that is used to determine whether or not to treat and/or how to treat. Imaging probe 100 can comprise a small profile (e.g. a distal portion with a diameter less than 0.034″) for accessing tight lesions, as described herein. System 10 can provide high resolution morphological images and other high-resolution morphological information, such as are produced using at least OCT data gathered by probe 100. In some embodiments, the high-resolution information produced by system 10 is based on OCT data and non-OCT data (e.g. at least angiography data).

In some embodiments, system 10 is configured to produce FFR information based on an OCT-derived lumen profile. The FFR information can also be based on side branch information. The FFR information can be based on combined OCT and non-OCT (e.g. angiography) information. The produced FFR information can be used (e.g. by a clinician of the patient) to decide whether or not to treat (e.g. whether or not to stent or otherwise treat a lesion).

In some embodiments, system 10 produces OCT image data (e.g. morphologic and/or lumen) and FFR data, and this combination of information is used (e.g. by a clinician) in planning a treatment procedure (including a decision of whether or not to treat). The provided information can also be based on non-OCT image data (e.g. angiography data) as described herein. The provided information can include morphological characteristics of a region of interest (e.g. a lesion), including the presence of calcium and/or lipids. The provided information can be used to determine which type of treatment should be performed (e.g. whether to perform an atherectomy procedure), and/or which size (e.g. diameter and/or length) of stent to implant. The provided information can include a prediction of results post-treatment (e.g. prediction of FFR post-treatment). For example, system 10 can be used to reduce delta FFR across a lesion and/or to achieve a desired FFR value (e.g. a value above 0.90).

In some embodiments, system 10 is used to treat multiple lesions (e.g. one or more serial lesions).

System 10 can utilize computational flow dynamics to model one or more lesions (e.g. each image using imaging probe 100 and/or second imaging device 15 of system 10), such as to determine which lesion to treat first. System 10 can be configured to provide information related to which of the one or more lesions to treat, such as when system 10 models each lesion's contribution to flow, such as to provide a clinician with information to decide which lesions to treat, and/or in which order to treat them.

In some embodiments, system 10 is configured to assess the outcome of a treatment of one or more lesions, such as when system 10 provides a quantitative or qualitative “score” of treatment success (e.g. efficacy and/or safety achieved via stenting and/or other treatment procedure). As described herein, such an assessment can include a post-treatment FFR calculation (e.g. including FFR value and/or delta FFR value), as well as post-treatment device assessment (e.g. an assessment of expansion, apposition, and/or position) and/or morphologic parameter assessment.

As described herein, system 10 is configured to predict the outcome of a treatment of one or more lesions, such as when system 10 provides a quantitative or qualitative “score” of predicted treatment success (e.g. efficacy and/or safety predicted to be achieved via stenting and/or other proposed treatment procedure). As described herein, such information can include a predicted post-treatment FFR calculation (e.g. including FFR value and/or delta FFR value), as well as a predicted post-treatment device assessment (e.g. a prediction of expansion, apposition, and/or position) and/or a morphologic parameter prediction. In some embodiments, if a prediction is below a threshold (e.g. a user or system 10 determined safety level), system 10 alerts the clinician of the potential issue. For example, system 10 can be configured to predict the likelihood of an acute event (e.g. an undesired acute event).

In some embodiments, system 10 is configured to provide blood vessel lumen contour images and/or other information, as described herein, such as when system 10 gathers OCT data and non-OCT data to produce the information. In some embodiments, system 10 utilizes one or more algorithms (e.g. algorithm 51), such as an algorithm utilizing artificial intelligence, to improve vessel contour information and/or other information determined by system 10. In some embodiments, algorithm 51 is configured to correct (e.g. allow a user to correct and/or automatically correct) blood vessel information. In some embodiments, system 10 is configured to alert a user of blood vessel information that is determined by algorithm 51 to have a low “confidence level”, such as when a portion of calculated blood vessel information has been identified by system 10 as potentially being not sufficiently accurate (e.g. has a confidence level below a threshold). This identification of potential issues allows a rapid correction and/or accuracy confirmation by a user. Correction and/or confirmation (“correction” herein) can be accomplished in various ways, such as: frame-by-frame, L-mode (e.g. longitudinal display) and/or other 2D view; and/or a 3D view.

In some embodiments, system 10 is configured to identify one or more side branches of the imaged vessel, such as when algorithm 51 is configured to identify side branches. Algorithm 51 can utilize artificial intelligence and/or user input to identify the side branches. In some embodiments, system 10 is configured to identify side branches based on both OCT data and non-OCT data (e.g. angiography data). In some embodiments, system 10 is configured to alert a user of calculated side branch information that is determined by algorithm 51 to have a low confidence level, as described herein. In some embodiments, ostial diameter of a side branch is calculated by system 10. Alternatively or additionally, side branch ostial diameter of a side branch can be input and/or corrected by a user.

In some embodiments, system 10 is configured to provide 3-D lumen information, as described herein. In these embodiments, system 10 can include a triggered pullback, such as a pullback that is triggered based on a patient physiologic parameter such as cardiac cycle and/or respiration. For example, triggering a pullback during a low motion phase of a cardiac cycle can be used to limit motion artifacts and/or improve accuracy of implant and/or vessel information.

In some embodiments, system 10 assumes flow velocity and/or other physiologic conditions to be constant in healthy vessels.

In some embodiments, system 10 is configured to alert the user if a pullback is inadequate (e.g. OCT data gathered during the pullback is inadequate), such as when system 10 identifies that the entire length of a lesion has not been captured.

In some embodiments, system 10 is configured to image tandem lesions and/or bifurcation lesions.

The final location of a pullback (e.g. the most-proximal location) can be within a delivery catheter 80 through which probe 100 has been inserted, such as to gather ostial information of the imaged vessel(s). In some embodiments, system 10 is configured to analyze OCT data to determine that a pullback has ended at a location within delivery catheter 80 (e.g. a distal portion of delivery catheter 80 has been imaged). In some embodiments, system 10 is configured to alert the user if the final pullback location (pre or post-pullback) is not within delivery catheter 80 (e.g. distal to the distal end of delivery catheter 80). For example, such an alert can be performed to prevent (if pre-pullback) or indicate (if post-pullback) an insufficient amount of ostial information will be or is included in the OCT-data. In some embodiments, system 10 is configured to automatically identify an imaged delivery catheter 80 (e.g. model information and/or construction information regarding delivery catheter 80). In some embodiments, system 10 is configured to “crop” (e.g. remove) the image of delivery catheter 80 from provided vessel images (e.g. to reduce file size, provide a simplified luminal view, and the like). In some embodiments, system 10 is configured to assess the positioning of the guide within the imaged vessel, such as when system 10 is configured to alert the user if “improper seating” of the guide is suspected.

In some embodiments, system 10 comprises pre-set configuration information, such as “image acquisition recipes” comprising one or more imaging parameter values (e.g. pullback distances and/or rates, a flush parameter, rotation rate, frame density, and/or other probe 100 use parameters) that is correlated to a particular situation, such as imaging of a particular artery or other blood vessel.

In some embodiments, system 10 is configured to assess a flush procedure that is performed while gathering OCT data (e.g. during a pullback). For example, system 10 can provide quantitative and/or qualitative information regarding the flush procedure, such as to alert a clinician if the assessment is below a threshold of acceptability (e.g. when system 10 applies a low confidence to collected image data due to an improper flush procedure). In some embodiments, FFR and/or other calculated information is not provided unless an adequate flush is determined by system 10.

In some embodiments, system 10 is configured to assess the presence of vessel spasm (e.g. during pullback or other image data collection period). For example, system 10 can, via algorithm 51, detect presence of spasm and alert the user. Alternatively or additionally, system 10 can apply a low confidence to collected image data due to the detected presence of spasm. In some embodiments, FFR and/or other calculated information is not provided when vessel spasm is determined to be present by system 10.

In some embodiments, system 10 is configured to assess the presence of thrombus (e.g. during pullback or other image data collection period). For example, system 10 can, via algorithm 51, detect presence of thrombus and alert the user. Alternatively or additionally, system 10 can apply a low confidence to collected image data due to the detected presence of thrombus. In some embodiments, FFR and/or other calculated information is not provided when thrombus is determined to be present by system 10.

In some embodiments, system 10 is configured to assess the presence of vessel dissection and/or other vessel injury (e.g. during pullback or other image data collection period). For example, system 10 can, via algorithm 51, detect presence of vessel injury and alert the user. Alternatively or additionally, system 10 can apply a low confidence to collected image data due to the detected presence of vessel injury. In some embodiments, FFR and/or other calculated information is not provided when vessel injury is determined to be present by system 10.

In some embodiments, system 10 is configured to assess the presence of a myocardial bridge (e.g. during pullback or other image data collection period). For example, system 10 can, via algorithm 51, detect presence of a myocardial bridge and alert the user. Alternatively or additionally, system 10 can apply a low confidence to collected image data due to the detected presence of a myocardial bridge. In some embodiments, FFR and/or other calculated information is not provided when a myocardial bridge is determined to be present by system 10.

In some embodiments, system 10 is configured to gather at least OCT data, such as when both OCT data and non-OCT data (e.g. angiography data) are recorded.

Subsequently, dynamic flow calculations can be performed, such as flow calculations performed at a low resolution (e.g. to reduce computational time). Simultaneously or subsequently, image corrections can be made (e.g. automatically by system 10 and/or manually by a user), such as lumen and/or side branch corrections. After corrections can be made, additional dynamic flow calculations can be performed, such as flow calculations at a high resolution.

Referring now to FIGS. 12 and 12A, a schematic view of the distal portion of an imaging probe and delivery catheter, and a magnified view of the components within circle M2, are illustrated, respectively, consistent with the present inventive concepts. Imaging probe 100 and delivery catheter 80 can be of similar construction and arrangement to imaging probe 100 and delivery catheter 80 described in reference to FIG. 1. Delivery catheter 80 includes lumen 84, through which imaging probe 100 has been slidingly inserted. In the embodiment shown in FIGS. 12-12A, a fluid pressurization mechanism, FPE 1500s shown, comprises a helical projection radially extending from optical core 110. During operation, as optical core 110 rotates, FPE 1500s rotates in unison, generating a fluid flow proximate FPE 1500s, and causing a pressure gradient within gel 118 (e.g. across FPE 1500s). Modeling of examples of the fluid flow dynamics are described herein in reference to FIGS. 12B-C.

In some embodiments, FPE 1500s comprises a helical coil, such as a spring or other wound wire, affixed along a portion of the length of optical core 110 (e.g. surrounding core 110). FPE 1500s can be adhered, such as via glue or another adhesive, to optical core 110. In some embodiments, FPE 1500s is molded on and/or with core 110, formed into (e.g. such as via a material removal process) core 110, fused onto core 110, and/or otherwise manufactured with or adhered to core 110. In some embodiments, FPE 1500s comprises a material selected from the group consisting of: metal; plastic; stainless steel; nickel-titanium alloy; nylon; polyether ether ketone (PEEK); polyimide; and combinations of these. In some embodiments, FPE 1500s can be formed directly onto optical core 110, such as using a deposition technique and/or 3D printing technique. In some embodiments, a selectively curable material is applied to optical core 110 and cured in a spiral pattern to form FPE 1500s. For example, a high-strength UV-cured adhesive can be applied to the surface of optical core 110 and selectively cured using a rotating, focused UV beam. In some embodiments, FPE 1500s can comprise a material selected to minimize deformation of FPE 1500s while the pressure gradient is applied. For example, during rotation a pressure gradient occurs across the length of FPE 1500s, such that to prevent deformation, a shorter FPE 1500s would require a stiffer material than a longer FPE 1500s configured to produce the same pressure gradient.

FPE 1500s comprises a radial height, height H₁, which is the distance from the surface of optical core 110 to the outer edge of FPE 1500s. Optical core 110 comprises a diameter D₁. Lumen 1205 of shaft 120 comprises an internal diameter D₂. In some embodiments, diameters D₁ and D₂ vary along the length of probe 100, and the following dimensions relate to a segment of probe 100, such as a distal segment shown in FIG. 12A (e.g. a segment proximal and proximate optical assembly 115). Probe 100 can comprise a clearance C₁, between FPE 1500s (e.g. the outer diameter of FPE 1500s) and the inner wall of shaft 120. Clearance C₁ relates both to the difference between diameter D₁ and D₂, and the height H₁ of FPE 1500s, such that C₁ equals one half of the difference between D₁ and D₂ minus H₁. In some embodiments, clearance C₁ comprises a clearance of no more than 100 μm, such as no more than 75 μm, such as between 10 μm and 75 μm. In some embodiments, height H₁ comprises a height that is between 5% and 95% of half the difference between D₁ and D₂, (e.g. a height H₁ that occupies at least 5% and/or no more than 95% of the space between the outer surface of core 110 and the inner wall of shaft 120). In some embodiments, the optimal height H₁ depends on factors such as: damping fluid viscosity (e.g. gel 118 viscosity); desired rotational rate of optical core 110; desired pressure gradient; and/or the clearance between FPE 1500s and the inner wall of shaft 120 (e.g. tighter clearances create higher pressures). In some embodiments, the coil profile of FPE 1500s comprises a width W1 as shown. Width W1 can comprise a width of 1% to 95% of diameter D₁. FPE 1500s can also comprise a pitch P1 as shown. Pitch P1 can comprise a pitch such that the gap between adjacent coils is 0.5 to 20 times the diameter D₁. In some embodiments, adjacent coils do not come into contact with each other. In some embodiments, pitch P1 is uniform along the length of FPE 1500s.

In some embodiments, gel 118 comprises a high viscosity, shear thinning fluid, such as is described in reference to FIG. 1. In some embodiments, the maximum functional clearance C₁ (e.g. the largest clearance C₁ allowable such that rotation of FPE 1500s generates sufficient fluid pressurization within lumen 1205), is proportional to the viscosity of gel 118. For example, the higher the viscosity of gel 118, the greater the maximum clearance C₁. In some embodiments, the clearance C₁ is proportional to the pressure differential that can be generated within gel 118 by rotating FPE 1500s, as described herein. For example, the smaller clearance C₁, the greater the pressure differential that can be generated. In some embodiments, clearance C₁ and height H₁ are minimized to limit turbulent, recirculatory, and/or other unwanted fluid flow proximate optical core 110. In some embodiments, gel 118 comprises a Newtonian (non-shear thinning) fluid. The dimensions C₁, H₁, D₁, and D₂ can be optimized for differing properties of gel 118.

In some embodiments, FPE 1500s comprises a covering (not shown). The covering can comprise a sheath, such as a heat shrink tube, and/or a painted or sprayed on coating. The covering can be configured to improve the bonding of FPE 1500s to optical core 110, and/or to control the dimensions of FPE 1500s (e.g. to hold FPE 1500s tightly to optical core 110, such as to limit unwanted variations in height H₁). Additionally or alternatively, the covering can be configured to modify the surface properties of either or both of optical core 110 and FPE 1500s. In some embodiments, the covering comprises a thickness that does not significantly affect the fluid propulsion and/or other fluid pressurization (“fluid pressurization” herein) performance of FPE 1500s. Alternatively or additionally, FPE 1500s can be constructed and arranged such that the dimensions C₁, H₁, and D₁ are optimal after the application of the covering.

In some embodiments, the pressurization of gel 118 within lumen 1205 caused by the rotation of FPE 1500s exerts a functional torsional sheer force on the inner wall of lumen 1205. Shaft 120 can comprise a torsional resistance greater than the functional torsional sheer force exerted by gel 118. In some embodiments, gel 118 exerts a torque of approximately. 004N-cm, and shaft 120 comprises a torsional resistance of at least 0.01N-cm, such as 0.03N-cm. Additionally or alternatively, FPE 1500s can exert a “wind-up” stress on optical core 110 as optical core 110 is rotated, driving FPE 1500s within gel 118. Optical core 110 can be constructed and arranged to not be adversely affected by (e.g. not break or otherwise fail) the sheer stress induced by the rotation of core 110 and FPE 1500, within gel 118 as well as the shear stress induced by the pullback motion within gel 118. In some embodiments, the additional windup stress on optical core 110 caused by FPE 1500s functions as a NURD reduction mechanism, similar to NURD reduction caused by gel 118, as described in applicant's co-pending International PCT Patent Application Serial Number PCT/US2018/062766, titled “Imaging System”, filed Nov. 28, 2018, the content of which is incorporated herein by reference in its entirety for all purposes.

Referring additionally to FIGS. 12B and 12C, a schematic view of the distal portion of an imaging probe showing a fluid flow pattern, and a fluid flow simulation, are illustrated, respectively, consistent with the present inventive concepts. The motion of gel 118 is depicted by fluid flow arrows FF in FIG. 12B, and paths FPp and FPD in FIG. 12C. Optical core 110 and FPE 1500s are depicted rotating with the top edge of FPE 1500s rotating into the page. Along with the axial motion shown, a fluid flow comprises a rotational component as well, shown in FIG. 12C. The distal end 1209 and/or at least a distal portion of shaft 120 can be sealed, such as when distal tip 119 comprises a cap or plug configured as a sealing element, plug 1209a shown in FIG. 12B. Rotation of FPE 1500s as shown causes a fluid flow proximate optical core 110 in the distal direction towards high pressure area HP. As the pressure within high pressure area HP increases to match the pressure of the distal fluid flow, a closed loop recirculation pattern emerges, as shown. Fluid propelled distally by FPE 1500s encounters the pressure within high pressure area HP and redirects proximally along the surface of lumen 1205 (e.g. along a path of least resistance). This fluid flow pattern creates a “dead head” pressure profile (e.g. there is no net fluid flow), maintaining the pressure gradient along FPE 1500s, from low pressure area LP to high pressure area HP. As shown in FIG. 12C, fluid paths FPD depict fluid flow proximate optical core 110, distally towards high pressure area HP. Fluid paths FPp depict fluid flow proximate the surface of lumen 1205, proximally towards low pressure area LP.

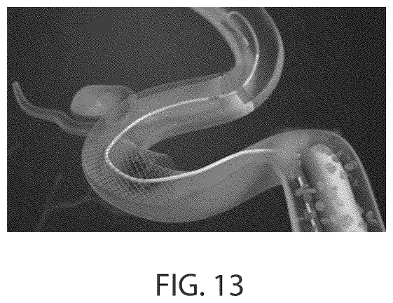

Applicant has conducted various studies using the systems, devices, and methods of the present inventive concepts. Results from some of these studies are described herein. Images and other graphics associated with those studies are shown in FIGS. 13-23.

In FIG. 13, principles of system 10 imaging in an intracranial vessel are illustrated. During a brief injection of contrast using a 5 F intermediate catheter, probe 100 is retracted while quickly rotating its internal optics resulting in a helical scanning pattern. By the means of a tightly spaced pattern (e.g., 60 μm) and an axial resolution approaching 10 μm, volumetric microscopy of the arterial wall, neurovascular devices and intraluminal objects is obtained.

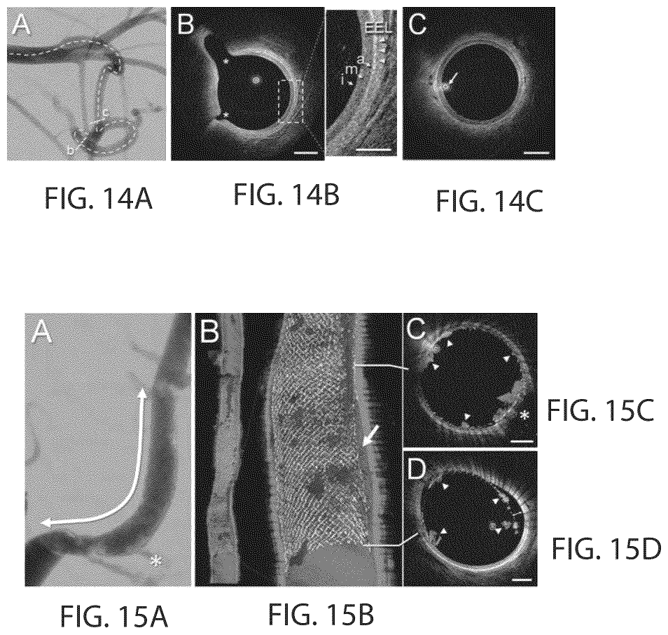

In FIG. 14, in vivo imaging in a flexed forelimb model of brachial porcine artery is shown. In portion (A), the dotted line highlights the tortuous path taken by probe 100 through the vessel. In portion (B), system 10 microscopy shows the external elastic lamina (arrowheads) and the individual layers of the vessel wall (arrows). A bright tunica intima is followed by a dark tunica media and a bright adventitia (inset). The asterisks denote the ostia of two side-branches, with diameters of 0.2 mm and 0.7 mm, respectively. In portion (C), the arrow indicates the eccentric position of probe 100 in the arterial lumen. The image shows a uniform illumination and absence of NURD artifact. Scale bars are equal to 1 mm (B-C), and 0.5 mm in the inset.

In FIG. 15, system 10 three-dimensional in vivo imaging of a stented swine internal maxillary artery is shown. In portion (A) vessel fluoroscopy is shown; in portion (B), system 10 three-dimensional cutaway rendering (top=distal; bottom=proximal) is shown. Flow-diverter malapposition (arrow) and clots of different sizes (purple color) are visible over the flow-diverter surface. In portion (C), cross-sectional system 10 imaging shows a jailed branch (indicated by the asterisk) and several thrombus formations over the flow-diverter surface (arrowheads). Portion (D) shows a flow-diverter proximal edge, with incomplete apposition (3 o'clock) and several clots over the device surface (arrowheads). Scale bars are equal to 1 mm. Three-dimensional rendering color scheme: red, artery wall; purple, clot; silver, metallic struts.

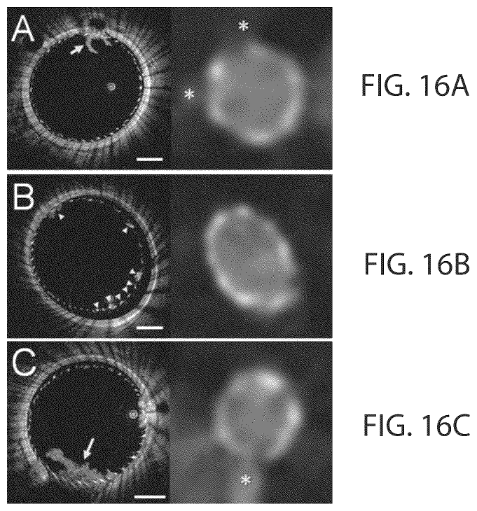

In FIG. 16, cross-sectional system 10 images compared to corresponding CBCT slices are shown. Portion (A) shows a side-branch thrombosis is visible on the system 10 image (arrow). Portion (B) shows flow-diverter malapposition with a maximum severity of approximately 400 μm is visible on the system 10 image between 1 and 8 o'clock. Small thrombus formations over the flow-diverter struts, with a thickness between 30 and 220 μm, are indicated by the arrowheads. Portion (C) shows thrombosis on the ostium of a large side-branch. The presence of thrombus and device malapposition are often undetected on the corresponding conebeam CT images. Scale bars are equal to 1 mm. The asterisk (*) on CT images denotes the location of side-branches.

FIG. 17 portions show: (A) an endoscopic view of system 10 volumetric microscopy data, where small, perforator-like side-branches jailed by an FDS are visible; (B) the side-branch located on the left side of the image (arrow) is free of clots and the FDS is well apposed to the parent artery; and (C) a second branch located on the right (indicated by the asterisk) shows FDS struts that are embedded by a clot. Scale bars are equal to 1.0 mm. Three-dimensional endoscopic rendering color scheme: red, artery wall; purple, clot; silver, metallic struts.

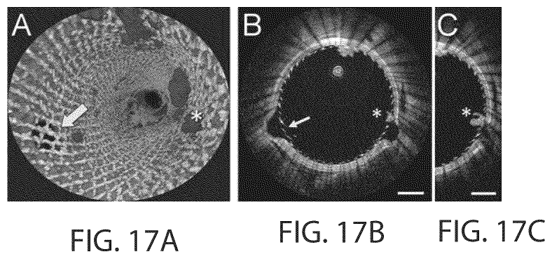

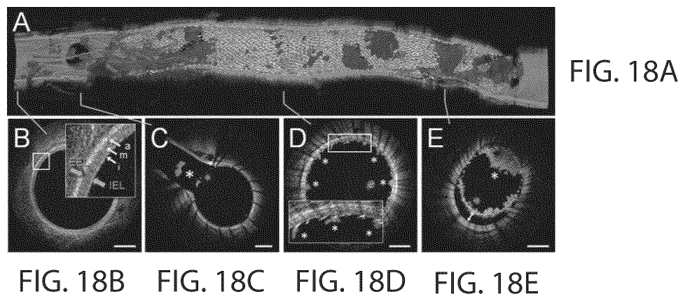

FIG. 18 portions (A) and (B) show: (A) system 10 volumetric microscopy data showing an ICS partially overlapping with the distal end of an FDS; (B) system 10 microscopy showing the individual vessel layers comprising a bright tunica intima, a low-scattering tunica media, and the tunica adventitia (arrows). The internal elastic lamina (IEL) and the external elastic lamina (EEL) are indicated by the green arrows. In portion (C), a thrombus dislodged from the surface of the device floating inside a large branch is visible and denoted by the asterisk. In portion (D), thrombus formations ranging between 100 μm and 200 μm in thickness are indicated by the asterisks and distributed over the FDS surface. In portion (E), a semi-occlusive clot formation in correspondence of a significant malapposition (at least 500 μm) is visible over the proximal end of the FDS. Scale bars are equal to 1.0 mm. Three-dimensional endoscopic rendering color scheme: red, artery wall; purple, clot; silver, flow-diverting stent struts; gray, neurovascular stent struts.

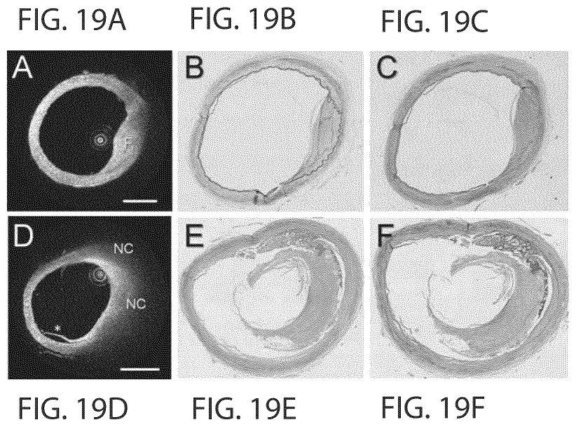

FIG. 19 shows intracranial plaques from an ex vivo segment of the MCA artery.

Portion (A) shows system 10 imaging of a fibrotic plaque and corresponding trichrome (B) and movat's staining (C). Fibrotic tissue is characterized by system 10 as a region of homogenous signal, resulting from elevated backscattering and low optical attenuation coefficients. In portion (D), system 10 imaging of a necrotic core plaque with corresponding H&E (E) and movat's staining (F). A necrotic core plaque is characterized on system 10 images as an area with poorly delineated borders followed by an elevated optical attenuation coefficient within an atherosclerotic plaque. The asterisk indicates a vessel wall dissection. Scale bars are equal to 1.0 mm.

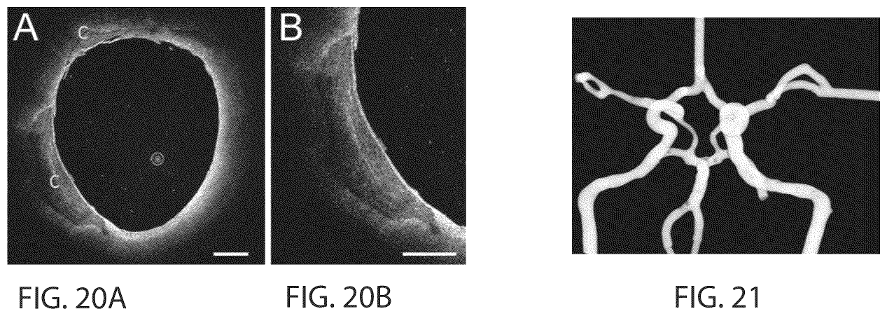

In FIG. 20, intracranial fibrocalcific plaques in a segment of an intradural vertebral artery are shown. In portion (A), two fibrocalcific plaques are visible in the image (each indicated by the letter C). Portion (B) is a magnified view of the bottom left portion of the vessel of portion (A). A calcific plaque with a thickness between 100 μm and 300 μm is located at 11 o'clock. A second plaque with a maximum thickness of 900 μm is located in the bottom-left quadrant of the image. Calcific tissue is characterized by a sharply demarcated area with a low and heterogeneous signal, resulting from low optical backscattering and absorption coefficients. Scale bars are equal to 1 mm.

In FIG. 21, a bench model of the full, patient-specific circle of Willis is shown. Merging of the divisions of the middle cerebral and anterior cerebral arteries bilaterally was performed to reduce the complexity of inputs and output of the flow circuit. For the same purpose, individual branch arteries such as the ophthalmic and superior cerebral arteries were removed.

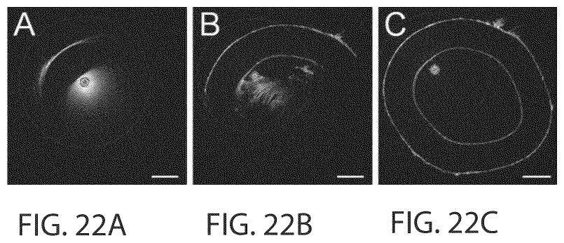

In FIG. 22, images of blood clearance testing are shown, by classifying the image into three different categories: (A) blood obscuring the field of view, (B) partial clearance, (C) complete clearance. In all cases, the contrast media injection rate was increased from a value of 1 ml/sec, using increments of 0.5 ml/sec, until a complete clearance state was detected. Scale bars are equal to 1 mm.

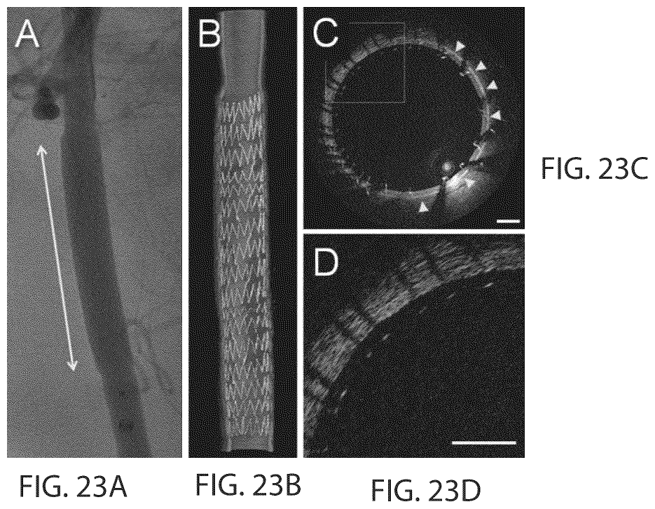

In FIG. 23, images of system 10 of a swine common carotid artery with maximum diameter of 5.9 mm following deployment of a Precise Pro Rx Carotid Stent System (Cordis) are shown. In portion (A), the stented segment is indicated by the arrow. Portion (B) shows three-dimensional rendering of a system 10 image. Portion (C) shows system 10 cross sectional imaging, illustrating the extended field-of-view of system 10 imaging technology. Even when probe 100 is located in an eccentric position within the arterial lumen, system 10 can visualize the entire stent and the arterial wall with sufficient brightness and illumination, as shown in portion (D). The arrowheads indicate the external elastic membrane (EEL). Scale bars are equal to 1.0 mm.

In current clinical practice, the endovascular treatment of stroke and other undesired cerebrovascular conditions is performed under the guidance of non-invasive imaging technology which is unable to provide sufficient resolution to adequately assess the underlying vessel pathology, device-vessel relationship, small perforating arteries and device related platelet aggregation. Intravascular, volumetric microscopy imaging performed using the optical probes and other system components of the present inventive concepts enables the patient's clinician to visualize the vessel wall microstructure and its interaction with neurovascular devices, and its use would have a profound impact in the treatment of cerebrovascular disease. System 10 is configured to rapidly acquire volumetric microscopy data at a resolution approaching 10 microns in highly tortuous vascular anatomy. Using system 10 in a combination of in vitro, ex vivo and in vivo models, its feasibility and efficacy for cerebrovascular microscopy has been demonstrated. Results of these studies has demonstrated the potential of system 10 for the imaging of intracranial arteries and for providing guidance for neuroendovascular therapeutic procedures.

Endovascular treatment of ruptured brain aneurysms results in less death and dependence as compared to surgery, with similar trends reported in the treatment of unruptured aneurysms. Self-expanding microstents and flow diverters, designed for the cerebrovasculature, with struts as small as 25 μm, have enabled the treatment of wide-neck, complex aneurysms. Given the limited x-ray attenuation of these devices on x-ray-based imaging systems, radiopaque markers are distributed along these treatment devices; however, it is currently not possible to properly image the entire device. This limitation is critically important since an accurate device placement with respect to the pathology and apposition are necessary for effective treatment and prevention of disabling complications. Despite tremendous advances in minimally invasive therapies, there currently remain limitations to endovascular treatments largely related to inadequate visualization of the device-vascular interface. Neurovascular treatment devices require particularly precise placement of the device to achieve the ultimate goal (e.g. complete exclusion of an aneurysm from the circulation). Moreover, endovascular thrombectomy (EVT) has become the standard of care for eligible patients suffering from ischemic stroke due to a large vessel occlusion. Non-invasive imaging techniques such as magnetic resonance (MR) vessel wall imaging have been proposed to assess vascular damage following EVT, however, there is insufficient resolution to directly visualize the underlying pathology (e.g., intracranial atherosclerotic disease or dissection), endothelial injury, and/or perforating arteries thrombosis.

Use of system 10 to perform volumetric microscopy, with the ability to visualize neurovascular devices in their entirety and the vessel wall microstructure in vivo, will have a profound impact in endovascular neurological treatments. Optical imaging techniques provided by system 10, with a resolution approaching the micron scale and feasible incorporation in small fiber-optics probes, are a promising candidate for a successful translation to clinical settings in which patients are treated. In the last decade, intracoronary optical coherence tomography (OCT) has become an increasingly popular modality, as it enables accurate measurements of the coronary lumen morphology, disease severity, and intracoronary stents. System 10 can be configured to provide an assessment of disease severity, such as an assessment that includes quantitative and/or qualitative assessment of disease severity. Beyond coronary applications, system 10 has the potential to revolutionize the diagnosis and management of cerebrovascular pathologies, such as: by imaging probe 100 providing the flexibility required to be advanced in tortuous cerebrovascular anatomy; by the ability of imaging probe 100 to perform imaging in elevated tortuosity; and/or by the components of system 10 being compatible with standard neurovascular clinical workflow. In addition, the enhanced field-of-view (i.e. acquired image diameter) of system 10 is sufficient to characterize large and complex carotid arteries and intracranial aneurysms. In applicant's studies, meeting of these cerebrovascular requirements was achieved using system 10.

### Results

In various studies, system 10 was used to perform volumetric microscopy in a combination of in vitro, in vivo and ex vivo models of cerebrovascular arteries. The imaging probe 100 used comprised a 0.016-inch wire-like catheter capable of being delivered through standard neurovascular microcatheters. Probe 100 is designed to have a comparable profile with state-of-the-art guidewires and features an atraumatic, radiopaque tip. The probe 100 optics have the ability to focus and collect the near-infrared light that is back-scattered by the vessel wall and intraluminal objects. Through a rapid rotation of optical assembly 115, and via retraction of probe 100 through the vessel, system 10 performs volumetric imaging of the surrounding artery and any implanted device at a resolution approaching 10 μm, reference FIG. 13. System 10 enables the acquisition of data at a frame rate of 250 images per second, with a field of view (i.e., image diameter) of at least 14 mm, from tortuous arterial segments longer than 50 mm, in 2-3 seconds.

### Vessel Clearing in an In Vitro Circulating Model of the Circle of Willis

Red blood cells scatter light and degrade its coherence properties and, as such, the acquisition of data by system 10 requires the displacement of the arterial blood from the vessel lumen. To test the ability to infuse sufficient contrast agent for the creation of a suitable optical window, simulated use experiments were performed in a patient-specific bench model of the complete circle of Willis, reference FIG. 21. Importantly, the model includes communicating arteries that have the potential to mix with injected contrast and obscure the acquisition of images by system 10. A pulsatile pump was used to circulate swine blood, and the optimal contrast injection protocols were identified at the location of the internal carotid (ICA), middle cerebral (MCA), basilar (BA) and vertebral (VA) arteries. For each of these locations, imaging probe 100 was delivered to the target anatomy and contrast media infused via a power injector through a 5F intracranial support catheter (5 F Navien, Medtronic Neurovascular, Irvine CA). Different concentrations of Omnipaque (GE Healthcare) radiopaque contrast were tested, including 240, 300 and 350 mgl/mL with a viscosity of 3.4, 6.3 and 10.4 cp at 37° C., respectively. Injections were performed using an automated pump (Medrad Mark 7 Arterion Injection System, Bayer HealthCare) flushing through the 0.058″ inner lumen of the intracranial support catheter at a pressure limit of 300 psi.

Clearance as a function of the contrast delivery rate was analyzed using a system 10 imaging metric by classifying the results in three categories: blood obscuring the field of view, partial clearance, and complete clearance, reference FIG. 22. Starting from 1 ml/see and using increments of 0.5 ml/sec, the flow rate of the contrast medium was increased until complete clearance was obtained and the optimal flushing protocol for each anatomical location determined. Omnipaque 350 was identified as the preferred media, having an optimal viscosity to efficiently displace the blood from the image field of view. Complete clearance of the ICA was obtained by flushing at 5 ml/sec and was observed after approximately 2-3 seconds of injection. Similarly, clearance of the MCA and the VA was obtained at a rate of 3 ml/sec. The BA required a 5 ml/sec injection to overcome the inflow from both VAs with the 5F catheter positioned at the level of the BA ostium. A rate of 4 ml/sec may be able to provide sufficient clearance when injecting contrast within a more distal position of the BA. System 10 data acquisitions with a duration of 2 seconds were shown to require injections for a minimum of 4 seconds to clear the artery, resulting in a 20 ml injection of contrast media for the ICA at 5 ml/sec and a 12 ml injection for the MCA at 3 ml/sec.

### Imaging in Elevated Vascular Tortuosity In Vivo

A flexed forelimb swine model (n=8) provided severe tortuosity conditions in brachial arteries, with a curvature similar to human ICA siphon. Imaging probe 100 was tested bilaterally in brachial arteries (n=16) acquiring system 10 image data sets with an effective imaging length of 65 mm+13 mm. The performance of the probe 100 devices was evaluated by assessing image artifacts that typically affect state-of-the-art intravascular technologies in tortuous anatomy, such as non-uniform rotational distortion (NURD). NURD is defined as an artifact occurring because of an increased friction on the rotating catheter components; it appears as smearing in the circumferential (rotational) direction, and it causes distortions that may lead to incorrect image interpretation and measurements. In brachial artery tortuosity (n=16), the system 10 data sets had no observed NURD artifacts. FIG. 14 shows an example of system 10 imaging. Angiography illustrates an elevated vascular tortuosity. Distortion-free imaging by system 10 with a uniform vessel wall illumination allowed precise visualization of the individual tissue layers, external elastic laminae (EEL), detailed lumen morphology and the ostia of two side-branches, reference FIG. 14B-C.

### Imaging of Neurovascular Stents and Flow-Diverters In Vivo

A comparison between digital subtraction angiography (DSA), contrast-enhanced conebeam CT (CBCT), and system 10 imaging was performed by acquiring data sets from swine internal maxillary arteries (IMAX, n=16), implanted with flow diverting stents (FDS, n=16) and self-expandable intracranial stents (ICS, n=15). Expert image readers (n=3) analyzed the presence of thrombus formations and malapposition using a binary outcome metric across the different modalities. Each reviewer analyzed a total of 480 images that included angiographic runs, system 10 and CBCT cross-sectional images. Fleiss' kappa statistic was used to assess the agreement between the different reviewers. An agreement was found of 0.90 (system 10), 0.67 (CBCT), and 0.49 (angiography) for the assessment of acute thrombus formation along the surface of the FDS. Values of 0.87 (system 10), 0.67 (CBCT), and 0.18 (angiography) for the diagnosis of device malapposition were found. A repeated analysis for the ICS device showed an agreement to visualize thrombus formation of 0.81 (system 10), 0.39 (CBCT) and 0.71 (angiography). Values of 0.78 (system 10), 0.45 (CBCT), and 0.41 (angiography) were found for ICS malapposition.

Examples of imaging data following FDS implantation are shown in FIG. 15. System 10 volumetric microscopy illustrates the presence of thrombus accumulations and of incomplete device apposition, reference FIG. 15B. A side-branch occlusion and FDS edge malapposition covered by multiple thrombus are detected, reference FIG. 15C-D. System 10 images and corresponding CBCT data are shown in FIG. 16 (system 10 images on the left, and CBCT data on the right). Side-branch thrombosis, reference FIG. 16A, malapposition of the device, and clots as small as 5 μm, 10 μm, or 30 μm, reference FIG. 16B, can be sufficiently captured by system 10 provided OCT images (e.g. identified by system 10 or by a clinician viewing images produced by system 10), and are often unseen (e.g. unidentifiable) on a corresponding CBCT cross-sections (e.g. as shown in FIGS. 16A-C). Further examples of the ability of system 10 images to depict neurovascular devices in vivo are shown in FIG. 17 and FIG. 18. Endoscopic data rendering illustrates two perforator-like branches that are jailed by an FDS, reference FIG. 17. The presence or absence of clots over their ostia is captured by system 10 images, reference FIG. 17B-C. Accurate visualization of the individual layers of the vessel wall, thrombus accumulation at the level of a side-branch jailed by an ICS, and small clots with a thickness of 100 μm are accurately captured by system 10 imaging, reference FIG. 18B-D. A partial vessel occlusion as a result of significant thrombosis over a malapposed FDS proximal edge is illustrated in FIG. 18E.

### Imaging in Large Arteries

To investigate the ability of system 10 to image large carotid arteries, stenting of common carotids was performed in a subgroup of the non-human animals (n=5). The diameter of the stented segments was found to be on average 5.5 mm+0.3 mm, with a maximum diameter of 5.9 mm. In all cases, the extended field of view of system 10 provided sufficient illumination to accurately assess the stent-vessel interaction on a strut level, reference FIG. 23.

### Imaging of Intracranial Atherosclerosis

Segments of diseased intracranial arteries (n=10) were obtained at autopsy from human patients older than 70 years of age with a history of vascular disease. Specimens were submerged in saline, and multiple system 10 image acquisitions were obtained to sample the entire length of each artery (n=15). Following the system 10 imaging procedure, an expert image reader identified a subset of the vessel segments containing representative examples of the three main plaque types using criteria previously established. Representative examples of fibrotic, fibrocalcific and necrotic core atherosclerotic disease were found (n=3). Tissue was processed by the means of histopathology techniques and stained using Sudan's black, Hematoxylin and Eosin (H&E), van Gieson's, Movat Pentachrome, and Trichrome reagents. A vascular pathologist blinded to system 10 imaging results analyzed the stained slices and characterized the plaques and tissue type. Agreement was found in all cases (n=3). Examples of intracranial plaques are shown in FIG. 19 and FIG. 20. A fibrotic plaque located in the M1 segment of the MCA of a cadaver of 97 years of age is shown in FIG. 19A. Using previously established criteria, a fibrotic plaque was identified on system 10 images as a thicker region of the artery wall exhibiting elevated optical backscattering and homogeneous intensity. Histopathology assessment categorized this plaque to be mostly composed of fibrotic tissue, reference FIG. 19B-C. A second plaque containing a necrotic core (NC) is illustrated in FIG. 19D. NCs are identified on system 10 images as signal-poor areas with poorly delineated boundaries within an atherosclerotic plaque covered by a fibrous cap. Necrotic tissue presents a strong optical attenuation, resulting in a fast image intensity signal drop-off, shadowing the vessel wall region located behind. Histopathology classified this plaque as fibrotic with a NC and underlying media degeneration, reference FIG. 19E-F. Lastly, a fibrocalcific plaque was identified in the distal segment of an intradural vertebral artery from a cadaver of 86 years of age. System 10 images display fibrocalcific plaques to contain evidence of fibrotic and calcific tissue, characterized by a signal-poor and heterogeneous region with sharply delineated borders. A fibrocalcific plaque, with a circumferential distribution of 87 degrees and a maximum thickness of approximately 900 μm, is shown in FIG. 20B. Calcific tissue presents low backscattering and attenuation coefficients in the near-infrared and, as a result, system 10 can accurately visualize both its thickness and circumferential distribution. Histopathology assessment categorized this plaque as mostly calcific, with evidence of underlying media degeneration and positive remodeling.

### DISCUSSION

Imaging performed using system 10 has the potential to provide sufficient spatial resolution to visualize details of neurovascular devices at a strut level that are unseen by state-of-the-art clinical imaging modalities. Previous research has shown the potential of intravascular imaging techniques for neurovascular applications; however, due to the mechanical characteristics of the existing coronary imaging catheters, their translation to tortuous cerebrovasculature was unfeasible. The data presented in applicant's studies illustrate how system 10 enables volumetric microscopy of intracranial arteries. Using a patient-specific in vitro model of the full circle of Willis, studies have presented contrast injection protocols that allow system 10 imaging at different neurovascular anatomical locations. The optimized injection protocols established for system 10 imaging do not differ from routine practice in neurointerventional surgery for the acquisition of rotational angiography, and in practice the acquisitions of these two different imaging modalities could occur simultaneously. In an in vivo non-human animal model, evidence was collected of the suitability of imaging probe 100 for imaging in elevated vascular tortuosity, comparable to the elevated tortuosity conditions encountered in the human ICA. Volumetric microscopy using system 10 resulted in much higher inter-operator agreement for the quantification of intraluminal clots and assessment of the interaction of neurovascular devices with the arterial wall, compared to state-of-the-art imaging modalities such as DSA and CBCT. Furthermore, existing criteria was translated for intravascular imaging to system 10 imaging, illustrating its ability to visualize the vessel wall microstructure and to characterize intracranial atherosclerosis. Taken together, the results of applicant's studies pave the way for the clinical translation of system 10 for intracranial imaging in the human clinical setting.

A growing body of pre-clinical evidence supports the speculation that cerebrovascular microscopy, with the ability to assess vessel wall disease and neurovascular devices, will have a profound impact on the endovascular treatment of cerebrovascular disease. Recently, it has been shown in a rabbit aneurysm model that the complete apposition of flow-diverting stents is critically important to achieve early and complete aneurysm occlusion, but histological evidence of poor apposition was not accurately captured on gold-standard DSA. Communicating malapposition between the aneurysm neck and flow-diverting devices, unseen on non-invasive modalities but captured by intravascular OCT, was shown to be a predictor of subsequent early aneurysm occlusion. Similarly, gaps in intrasaccular device reconstruction of aneurysm necks, observed by system 10 images and undetected on CBCT, were shown to correlate with subsequent lack of occlusion. MRVW is the method of choice for intracranial vessel wall imaging in the context of ischemic stroke and intracranial hemorrhage; however its accuracy is significantly affected by a limited spatial resolution and voxel size. A recent study showed that high-resolution intravascular imaging was able to reveal endothelial injuries, residual thrombus and ongoing thrombosis on basilar artery perforators at the lesion site following EVT, in cases where angiographic techniques showed a complete recanalization and no clots were visible on CT angiography and MRVW.

System 10 enables volumetric microscopy of the arterial wall at a resolution approaching 10 μm. In applicant's studies, it has been shown in an animal model that system 10 can accurately identify intraluminal thrombus and neurovascular devices at a strut level. These results, in combination with the evidence provided for imaging in tortuous anatomy, suggest that system 10 can be used clinically for peri-procedural assessment of neurovascular devices so that corrective measures, such as local administration of GP IIb/IIIa inhibitors, angioplasty or additional stenting, can be deployed. In cases where angiographic findings are ambiguous, the superior resolution of system 10 imaging allows the visualization of vessel dissections and stent-vessel interactions which non-invasive imaging techniques are unable to capture. An accurate characterization of intracranial atherosclerotic plaque type may inform treatment decisions as well as enable improved stent sizing, placement and interaction with perforating arteries and residual stenosis for improved treatment of intracranial arteries. System 10 may enable more informed, image guided, personalized antithrombotic management after EVT. The superior resolution of system 10 images provides the ability to study the vessel and aneurysm healing response to an implanted device at an unprecedented level, assessing the tissue thickness over the device surface and vascular remodeling with precision and accuracy of microscopy techniques in vivo. As such, the use of system 10 provided cerebrovascular microscopy offers great potential for monitoring device healing in clinical practice, assessing endothelial overgrowth and intimal hyperplasia, and offering insights for optimal antiplatelet therapy following endovascular treatments. In the pre-clinical setting, system 10 will provide insights supporting the development of novel generations of neurovascular devices.

### Study Limitations

The ability to inject contrast media and achieve a clear optical window for imaging of neurovascular arteries was investigated in a bench model of the circle of Willis. Substantial efforts were made to reproduce patient circulation, including pulsatile blood flow, physiological flow rates, vessel caliber, and capillary resistance. However, the use of in vitro models may be unable to capture the large spectrum of anatomical variations and disease conditions encountered in patient cerebrovasculature. In applicant's studies, a higher viscosity agent was more effective in successfully displacing blood and obtaining a clear field-of-view. In human clinical practice, other factors such as intracranial artery disease, aging, and consequent reduction of blood flow may permit the use of lower viscosity agents, such as saline or reduced viscosity contrast. In these conditions, injection at lower rates may also be possible and provide sufficient clearance for system 10 image acquisitions. Furthermore, the use of low molecular weight dextran has been successfully demonstrated for coronary imaging, its safety investigated for intracranial use, and future studies may possibly explore its use for system 10 imaging.

### Patient-Specific Vascular Model of the Circle of Willis

A patient-specific vascular model including the entire circle of Willis was used to investigate optimal blood clearing protocols at different anatomical locations including the ICA, the MCA, the intradural VA, and the basilar arteries, reference FIG. 21. This model has been previously used in similar studies. Multiple sensors were used in applicant's studies to constantly measure the ICA, MCA, and BA flow rates and pressure to ensure reproduction of clinically relevant values. Swine blood was maintained at 37° C. and circulated using a pulsatile pump. The resistance of the outlets was adjusted to achieve physiologically representative flow rates through each branch of the model. Blood pressure and flow rate were constantly monitored and adjusted to maintain physiological values of approximately 250 ml/min in the ICA, 140 ml/min in the MCA, and 160 ml/min in the BA.

### Animal Model and Preparation

A Yorkshire swine model ranging between 40-70 kg (n=8) was used in applicant's studies. All procedures were performed under general anesthesia. The animals were pre-anesthetized by a subcuticular injection of glycopyrrolate (0.01 mg/kg). Anesthesia was induced by an intramuscular injection of tiletamine (Telazol, 4.4 mg/kg), ketamine (2.2 mg/kg), and xylazine (2.2 mg/kg) and was maintained through mechanical ventilation of 1-3% isoflurane. During the procedures, the following vital parameters were monitored continuously and recorded: heart and respiratory rate, invasive blood pressure, oxygen saturation, end-tidal CO2, and temperature.

A model of elevated vascular tortuosity was obtained by the means of a flexed forelimb model. This technique is able to provide severe tortuosity in brachial arteries, resulting in radii of curvature similar to the ones encountered in the human ICA. After the surgical exposure of the right femoral artery, a 10F introducer sheath was inserted for endovascular access and a 0.058-inch Navien™ (Medtronic) was navigated through the proximal segment of the brachial artery allowing the deployment of imaging probe 100 to the target anatomy.

The 0.058-inch intracranial support catheter was subsequently navigated into the internal maxillary artery (IMAX). Sixteen (n=16) FDS were deployed bilaterally into the IMAX. A Pipeline FDS (Medtronic) was used in half of the arteries (n=8), and a Surpass FDS (Stryker neurovascular) was implanted in the other half (n=8) by the means of conventional endovascular surgery techniques. Following FDS implantation, an ICS was deployed in each IMAX resulting in a partially overlapping segment. Wingspan stents (Stryker Neurovascular) were implanted in a total of 5 arteries, Neuroform stents (Stryker Neurovascular) in 6 arteries, and Solitaire AB stents (Medtronic) in 4 arteries, for a total of 15 devices. In one single case, no ICS was deployed due to a thrombotic occlusion of the artery at the distal end of the FDS. In addition, a Precise Pro Carotid stent system (Cordis) was implanted in a subset of the animals in the common carotid artery (n=5).

### Acquisition of Imaging Data

System 10 imaging was performed bilaterally in swine brachial arteries of all non-human animals (n=16). Contrast media (Omnipaque) was injected by the means of a 5 F intermediate catheter to displace blood from the arterial lumen. Intravascular system 10 images were acquired in all IMAX arteries, following the deployment of FDS and ICS devices, obtaining a total of 31 imaging runs. Sixteen (n=16) of these imaging data sets were acquired following the FDS implantation. The additional datasets (n=15) were obtained following the ICS implantation partially overlapping with the FDS. Similarly, digital subtraction angiography (DSA), non-subtracted cineangiography, and full-scale, small FOV cone-beam CT images (Philips Healthcare) were acquired for each vessel using standard imaging techniques, obtaining 31 imaging runs for each modality.

### Analysis of Neurovascular Devices

Co-registration of the imaging data from the different modalities was obtained using the distal and proximal edges of the FDS and ICS as reference markers. Five millimeter (5 mm) long regions of interests (ROI) were defined at the proximal and distal edges of all devices. For each ROI, the locations containing the largest clot and the most severe malapposition were matched between DSA, system 10, and the reconstructed CBCT cross-sectional images. A binary scoring system was used to classify all images for the presence or absence of clots and device malapposition. Altogether, 160 angiographic projections, 160 System 10 images, and 160 CBCT cross-sectional images were analyzed by three experienced neurointerventionalists in a blinded fashion.

### Ex Vivo Segments of Intracranial Artery

To compare system 10 imaging of intracranial atherosclerotic plaques with histopathology, arterial segments were obtained from cadavers older than 70 years of age with a history of smoking and coronary and/or peripheral artery disease. The intracranial vasculature of three cadavers was explored, and arteries appearing to contain atherosclerotic disease were harvested (n=10), including distal segments of the ICA (n=2), proximal and distal MCA (n=4), BA (n=2) and intradural VA (n=2). Specimens were traditionally fixated with a 10% formalin solution prior to being harvested and subsequently the specimens were submerged in saline for system 10 image acquisitions. Multiple data sets sampling the entire length of each artery were obtained. An expert system 10 image reader identified regions of interest (ROI) containing atherosclerotic disease using previously established criteria for fibrotic, fibrocalcific and necrotic core plaques (n=3). A visible light indicator emitted by image probe 100 was used to mark each ROI on the different specimens. The corresponding tissue samples were subsequently embedded in paraffin, sectioned in 5 μm slices, and processed for Sudan's black, Hematoxylin and Eosin, van Gieson's, Movat Pentachrome, and Trichrome staining.

### Statistical Analysis

Unless otherwise specified, data are presented as mean±standard deviation. Fleiss' kappa statistics was used to assess and quantify the agreement between the three different image raters classifying DSA, CBCT and system 10 images (inter-operator variability).

### Three-Dimensional System 10 Renderings

Cross-sectional images were segmented following previously established criteria (13) using software ImageJ (36). Intraluminal clots, struts of neurovascular devices, and the contour of the vessel wall were manually traced and labelled. System 10 data were rendered in color using the following scheme: red, artery wall; purple, intraluminal clots; silver, metallic struts. The segmented data sets were imported into a volume-rendering DICOM visualization software (OsiriX MD v10.0.2, Pixmeo SARL, Bernex, Switzerland), after an automatic frame-to-frame registration to correct for motion artifacts generated by the mechanical scanning of the catheter. Perspective volume rendering techniques using cut surfaces and fly-through methods with different opacity tables were used for visualization, similar to previous studies (14, 37).

The above-described embodiments should be understood to serve only as illustrative examples; further embodiments are envisaged. Any feature described herein in relation to any one embodiment may be used alone, or in combination with other features described, and may also be used in combination with one or more features of any other of the embodiments, or any combination of any other of the embodiments. Furthermore, equivalents and modifications not described above may also be employed without departing from the scope of the invention, which is defined in the accompanying claims.

1. (canceled)

2. A method of imaging a blood vessel within a patient, the method comprising:
(a) inserting an imaging probe into the blood vessel, the imaging probe comprising:
an elongate shaft comprising a proximal end, a distal portion comprising an outer diameter no greater than 2.6 Fr (0.034″), and a lumen extending between the proximal end and the distal portion;
an optical core comprising a proximal end and a distal end, wherein at least a portion of the rotatable optical core is positioned within the lumen of the elongate shaft; and
an optical assembly positioned proximate the distal end of the optical core;
(b) directing light from a light source having a coherence length greater than 10 mm to the optical assembly, and receiving reflected light from tissue at the optical assembly;
(c) rotating the optical core to scan tissue over a scan range of no less than 6 mm; and
(d) acquiring intravascular imaging data based on the reflected light.

2. The method of claim 2, wherein the outer diameter of the distal portion of the elongate shaft is no greater than 2.0 Fr (0.026″).

3. The method of claim 2, wherein the outer diameter of the distal portion of the elongate shaft is no greater than 1.7 Fr (0.022″).

4. The method of claim 2, further comprising calculating an FFR value based on the intravascular imaging data.

5. The method of claim 2, wherein step (d) acquiring intravascular imaging data is performed during a pullback procedure, wherein the optical core is retracted at a rate of at least 25 mm/sec.

6. The method of claim 5, wherein the pullback procedure is performed during a resting portion of a heart cycle to minimize motion artifacts.

7. The method of claim 5, wherein a pullback distance of the pullback procedure is at least 10 cm and the pullback procedure is performed in a time period of no more than 4 seconds.

8. The method of claim 2, further comprising:
(e) receiving extravascular imaging data of the blood vessel from a second imaging device, wherein the extravascular imaging data comprises angiography data; and
(f) co-registering the intravascular imaging data with the angiography data.

9. The method of claim 8, wherein step (f) co-registering the intravascular imaging data with the angiography data comprises:
identifying one or more side branches of the blood vessel in the intravascular imaging data;
identifying the one or more side branches in the angiography data; and
aligning the intravascular imaging data and the angiography data based on the one or more identified side branches.

10. The method of claim 8, wherein step (f) co-registering the intravascular imaging data with the angiography data comprises:
identifying a location of one or more radiopaque markers of the imaging probe in the angiography data;
identifying the one or more radiopaque markers in the acquired imaging data; and
aligning the intravascular imaging data and the angiography data based on the one or more identified radiopaque markers.

11. The method of claim 2, further comprising providing treatment information based on the intravascular imaging data.

12. The method of claim 11, further comprising performing a post-treatment imaging procedure on the patient based on the treatment information, and acquiring a second set of intravascular imaging data after the post-treatment imaging procedure has been performed.

13. The method of claim 2, further comprising generating a longitudinal representation of the blood vessel based on the intravascular imaging data.

14. The method of claim 13, further comprising:
identifying a plurality of vessel features in the longitudinal representation;
color-coding the longitudinal representation based on the identified vessel features; and
displaying the color-coded longitudinal representation to an operator.

15. The method of claim 2, further comprising:
calculating a lumen cross-sectional area based on the intravascular imaging data; and
calculating flow dynamics of the blood vessel based on the lumen cross-sectional area.

16. The method of claim 15, wherein the calculated flow dynamics include a fractional flow reserve (FFR) value.

17. The method of claim 2, wherein the method is performed to image a lesion comprising a diameter of less than 0.080″.

18. The method of claim 2, further comprising analyzing the intravascular imaging data to identify one or more of: lumen boundaries; side branches; healthy portions of lumen; diseased portions of lumen; type of disease imaged; or a location of a guidewire.

19. The method of claim 2, wherein the intravascular imaging data is OCT data.

20. The method of claim 2, further comprising imaging a clot of 5 μm or more.

21. The method of claim 2, wherein the optical core is rotated at a rate of at least 250 rotations per second.
